(19) United States
(12) Patent Application Publication
(10) Pub. No.: US 2024/0141436 A1
(43) Pub. Date: May 2, 2024

(54) Compounds, Compositions and Methods of Treatment Thereof

(71) Applicant: iTeos Belgium SA, Gosselies (BE)

(72) Inventors: Chiara Martinoli, Milan (IT); Noemie Wald, Brussels (BE); Shouraydeep Srivastava, Andover, MA (US); Joanne Lager, Hollis, NH (US); Sally Ross, Cheadle (GB)

(73) Assignee: iTeos Belgium SA, Gosselies (BE)

(21) Appl. No.: 18/277,256

(22) PCT Filed: Feb. 17, 2022
(86) PCT No.: PCT/US2022/016808

§ 371 Date: Aug. 15, 2023

Related U.S. Application Data
(60) Provisional application No. 63/150,522, filed on Feb. 17, 2021.

(51) Int. Cl. C12Q 1/6886; A61K 31/519; A61P 35/00

(52) U.S. Cl. CPC C12Q 1/6886; A61K 31/519; A61P 35/00; C12Q 2600/106; C12Q 2600/158

(57) ABSTRACT

The present disclosure relates to treatment of a subject who has been identified with an increased A_{2A} expression or density of A_{2A}R⁺ cells.

### FIELD OF THE PRESENT DISCLOSURE

The present disclosure includes treatment of a subject wherein the subject has been identified as having tumor-infiltrating immune cells (TWO with high levels of A_{2A} receptor (A_{2A}R) expression. Compounds of the present disclosure include, but are not limited to A_{2A}R antagonist, and are useful as therapeutic compounds, especially in the treatment of cancers. Compounds of the present disclosure may be used in combination with additional therapeutic agents for the treatment of cancer.

### BACKGROUND

Many tumors produce high levels of extracellular adenosine which suppress anti-tumor immune responses. Blocking A_{2A} receptors, predominantly expressed on tumor-infiltrating immune cells, can reverse the immunosuppressive effect of adenosine. Inupadenant is designed as a non brain-penetrant potent and highly selective small molecule antagonist of the A_{2A} receptor that remains active even at the high adenosine concentrations found in tumors.

### SUMMARY

The present disclosure includes a method of treating cancer characterized by high levels of A_{2A} receptor expression in a subject in need thereof, comprising administering to the subject a therapeutically effective amount of a A_{2A}R antagonist, or a combination thereof.

The present disclosure is further defined in the appended claims.

### BRIEF DESCRIPTION OF THE DRAWINGS

FIG. 1 comprises three graphs: a graph showing the A_{2A}R⁺ cells/mm² in and PR+SD patients, an ROC curve comparing sensitivity % to specificity %, and percent change in tumor size from baseline as described in Example 1.

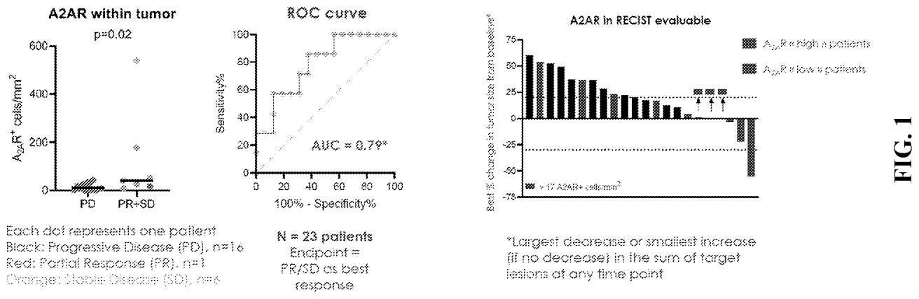

FIG. 2A is a box and whisker plot of baseline ADORA_{2A} expression and FIG. 28 is a box and whisker plot of density of A_{2A}R⁺ cells within the tumor area, each of which in subjects who had partial response or non-progressive disease described in Example 2.

FIG. 3 comprises three graphs discussed in Example 2: (FIG. 3A) an ROC curve comparing sensitivity % to specificity %, (FIG. 3B) Survival analysis by A_{2A}R cell density using the optimized cut-off, and (FIG. 3C) Waterfall plot of best percent change in the sum of target lesion diameters in RECIST-evaluable subjects.

FIG. 4 comprises two panels discussed in Example 3: (FIG. 4A) ADORA₂A expression analyzed by gRT-PCR in six human tumor cell lines, and (FIG. 4B) A_{2A}R expression assessed by Western blot in six human tumor cell lines.

FIG. 5 comprises two panels: A_{2A}R expression was assessed by flow cytometry in HEK-293 wild-type (empty histograms) or overexpressing A_{2A}R (filled histograms) using an anti-A_{2A}R antibody (FIG. 5A), or A_{2A}R probe (FIG. 5B).

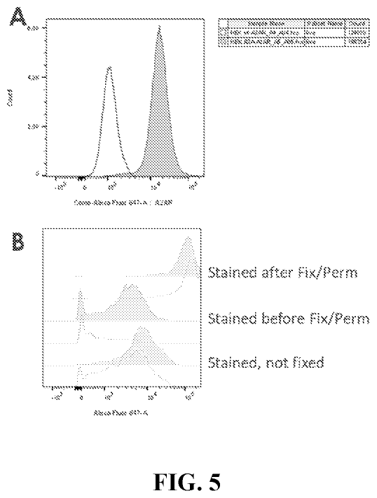

FIG. 6 comprises two panels described in Example 4: A_{2A}R⁺ cells (FIG. 6A) and ADORA₂A expression (FIG. 6B) in tumor tissue samples. A: Representative image of a tissue section co-stained with anti-A_{2A}R and anti-CD3 antibodies. B: Representative image of CISH on a tissue section. Arrows indicate ADORA_{2A} RNA molecules.

### DEFINITIONS

In the present disclosure, the following terms have the following meanings:

The teens “adenosine A_{2A} receptor” “A_{2A} receptor” and “A_{2A}R” are used interchangeably to refer to a cell surface adenosine receptor with adenosine as the endogenous ligand. In human, A_{2A}R is encoded by the ADORA2A gene. An exemplary amino acid sequence of human A_{2A}R includes SEQ ID NO: 1.

| SEQ ID | Sequence |
|---|---|
| SEQ ID No. 1 | MPIMGSSVYITVELAIAVLAILGNVLVCWAVWLNSN |
| | LQNVTNYFVVSLAAADIAVGVLAIPFAITISTGFCAAC |
| | HGCLFIACFVLVLTQSSIFSLLAIAIDRYIAIRIPLRYNG |
| | LVTGTRAKGIIAICWVLSFAIGLTPMLGWNNCGQPKE |
| | GKNHSQGCGEGQVACLFEDVVPMNYMVYFNFFACV |
| | LVPLLLMLGVYLRIFLAARRQLKQMESQPLPGERARS |
| | TLQKEVHAAKSLAIIVGLFALCWLPLHIINCFTFFCPDC |
| | SHAPLWLMYLAIVLSHTNSVVNPFIYAYRIREFRQTFR |
| | KIIRSHVLRQQEPFKAAGTSARVLAAHGSDGEQVSLR |
| | LNGHPPGVWANGSAPHPERRPNGYALGLVSGGSAQE |
| | SQGNTGLPDVELLSHELKGVCPEPPGLDDPLAQDGAG |
| | VS |

The term “aldehyde” refers to a group —CHO.

The term “alkenyl” refers to unsaturated hydrocarbyl group, which may be linear or branched, comprising one or more carbon-carbon double bonds. Suitable alkenyl groups comprise between 2 and 6 carbon atoms, preferably between 2 and 4 carbon atoms, still more preferably between 2 and 3 carbon atoms. Examples of alkenyl groups are ethenyl, 2-propenyl, 2-butenyl, 3-butenyl, 2-pentenyl and its isomers, 2-hexenyl and its isomers, 2,4-pentadienyl and the like.

The term “alkenylcarbonyl” refers to a group —(C═O)-alkenyl wherein alkenyl is as herein defined.

The term “alkenylcarbonylamino” refers to a group —NH—(C═O)-alkenyl wherein alkenyl is as herein defined.

The term “alkoxy” refers to a group —O-alkyl wherein alkyl is as herein defined.

The term “alkyl” refers to a hydrocarbyl radical of formula CₙH₂ₙ₊₁ wherein n is a number greater than or equal to 1. Generally, groups of this disclosure comprise from 1 to 8 carbon atoms, more preferably, alkyl groups of this disclosure comprise from 1 to 6 carbon atoms. Alkyl groups may be linear or branched. Suitable alkyl groups include methyl ethyl, propyl, butyl, pentyl, hexyl, heptyl and octyl.

The term “alkylaminoalkyl” refers to a group -alkyl-NH-alkyl wherein alkyl is as herein defined.

The term “alklaminoalkylaminocarbonyl” refers to a group —(C═O)—NH-alkyl-NH-alkyl wherein alkyl is as herein defined.

The term “(alkylaminoalkyl)(alkyl)aminocarbonyl” refers to a group —(C═O)—NR¹R² wherein R¹ is an alkyl group and R² is a -alkyl-NH-alkyl group, wherein alkyl is as herein defined.

The term “alkylaminoalkylcarbonyl” refers to a group —(C═O)-alkyl-NH-alkyl wherein alkyl is as herein defined.

The term “alkylcarbonyl” refers to a group —(C═O)-alkyl wherein alkyl is as herein defined.

The term “alkylcarbonylamine” refers to a group —NH—(C═O)-alkyl wherein alkyl is as herein defined.

The term “alkylcarbonyloxyalkyl” refers to a group -alkyl-O—(C═O)-alkyl wherein alkyl is as herein defined.

The term “alkylheteroaryl” refers to any heteroaryl substituted by an alkyl group wherein alkyl is as herein defined.

The term “alkyloxyalkyl” refers to a group -alkyl-O-alkyl wherein alkyl is as herein defined.

The term “alkyloxyalkyloxy” refers to a group —O-alkyl-O-alkyl wherein alkyl is as herein defined.

The term “alkyloxycarbonyl” refers to a group —(C═O)—O-alkyl wherein alkyl is as herein defined.

The term “alkylsulfonyl” refers to a group —SO₂-alkyl wherein alkyl is as herein defined.

The term “alkylsulfonylaminoalkyl” refers to a group -alkyl-NH—SO₂-alkyl wherein alkyl is as herein defined.

The term “alkylsulfonealkyl” refers to a group -alkyl-SO₂-alkyl wherein alkyl is as herein defined.

The term “alkylsulfonimidoyl” refers to a group —S(═O)(═NH)-alkyl wherein alkyl is as herein defined.

The term “alkylsulfoxide” refers to a group —(S═O)-alkyl wherein alkyl is as herein defined.

The term “alkylsulfoxidealkyl” refers to a group -alkyl-SO-alkyl wherein alkyl is as herein defined.

The term “alkyne” refers to a class of monovalent unsaturated hydrocarbyl groups, wherein the unsaturation arises from the presence of one or more carbon-carbon triple bonds. Alkynyl groups typically, and preferably, have the same number of carbon atoms as described above in relation to alkyl groups. Non-limiting examples of alkynyl groups are ethynyl, 2-propynyl, 2-butynyl, 3-butynyl, 2-pentynyl and its isomers, 2-hexynyl and its isomers and the like.

The term “alkynealkyl” refers to a group -alkyl-alkyne wherein alkyl and alkyne are as herein defined.

The term “amino” refers to a group —NH₂.

The term “aminoalkyl” refers to a group -alkyl-NH₂ wherein alkyl is as herein defined.

The term “aminoalkylaminocarbonyl” refers to a group —(C═O)—NH-alkyl-NH₂ wherein alkyl is as herein defined.

The term “aminoalkylcarbonylamino” refers to a group —NH—(C═O)-alkyl-NH₂ wherein alkyl is as herein defined.

The term “aminocarbonyl” or “aminocarboxy” refers to a group —(C═O)—NH₂.

The term “(aminocarbonylalkyl)(alkyl)amino” refers to a group —NR¹R² wherein R¹ is an alkyl group and R² is a -alkyl-(C═O)—NH₂ group, wherein alkyl is as herein defined.

The term “aminocarbonylalkylamino” refers to a group —NH-alkyl-(C═O)—NH₂ wherein alkyl is as herein defined.

The term “aminosulfonyl” refers to a group —SO₂—NH₂.

The term “aryl” refers to a polyunsaturated, aromatic hydrocarbyl group having a single ring (i.e. phenyl) or multiple aromatic rings fused together (e.g. naphtyl), typically containing 5 to 12 atoms; preferably 5 to 10; more preferably the aryl is a 5- or 6-membered aryl. Non-limiting examples of aryl comprise phenyl, naphthalenyl.

The term “arylalkyl” refers to a group -alkyl-aryl wherein alkyl and aryl are as herein defined.

The term “aryloxyalkyl” refers to a group -alkyl-O-aryl wherein alkyl and aryl are as herein defined.

The term “carbonyl” refers to a group —(C═O)—.

The term “carbonylamino” refers to a group —NH—(C═O)—.

The term “cyano” refers to a group —CN.

The term “cyano” refers to a group -alkyl-CN wherein alkyl is as herein defined.

The term “cycloalkyl” refers to a cyclic alkyl group, that is to say, a monovalent, saturated, or unsaturated hydrocarbyl group having 1 or 2 cyclic structures. Cycloalkyl includes monocyclic or bicyclic hydrocarbyl groups. Cycloalkyl groups may comprise 3 or more carbon atoms in the ring and generally, according to this disclosure comprise from 3 to 10, more preferably from 3 to 8 carbon atoms; still more preferably more preferably the cycloalkyl is a 5- or 6-membered cycloalkyl. Examples of cycloalkyl groups include but are not limited to cycloproryl, cyclobutyl, cyclopentyl, cyclohexyl.

The term “cycloalkyloxy” refers to a group —O-cycloalkyl wherein cycloalkyl is as herein defined.

The term “dialkylamino” refers to a group —NR¹R² wherein R¹ and R² are both independently alkyl group as herein defined.

The term “dialkylaminoalkyl” refers to a group -alkyl-NR¹R² wherein R¹ and R² are both independently alkyl group, as herein defined.

The term “dialkylaminoalkylaminocarbonyl” refers to a group —(C═O)—NH-alkyl-NR¹R² wherein R¹ and R² are both alkyl group, as herein defined.

The term “dialkylaminoalkylcarbonyl” refers to a group —(C═O)-alkyl-NR¹R² wherein R¹ and R² are both alkyl group, as herein defined.

The term “dihydroxyalkyl” refers to a group alkyl is as herein defined substituted by two hydroxyl (—OH) groups.

The term “halo” or “halogen” refers to fluoro, chloro, bronco, or iodo.

The term “haloalkyl” refers to an group in which one or more hydrogen atom is replace by a halogen atom.

The term “haloalkyloxy” refers to a group —O-haloalkyl wherein alkyl is as herein defined.

The term “heteroaryl” refers to an aryl group as herein defined wherein at least one carbon atom is replaced with a heteroatom. In other words, it refers to 5 to 12 carbon-atom aromatic single rings or ring systems containing 2 rings which are fused together, typically containing 5 to 6 atoms; in which one or more carbon atoms is replaced by oxygen, nitrogen and/or sulfur atoms where the nitrogen and sulfur heteroatoms may optionally be oxidized and the nitrogen heteroatoms may optionally be quaternized. Non-limiting examples of such heteroaryl, include: pyrrolyl, furanyl, thiophenyl, pyrazolyl, imidazolyl, oxazolyl, isoxazotyl, thiazobyl, isothiazolyl, triazobyl, oxadiazolyl, thiadiazolyl, pyridinyl, pyridazinyl, pyrimidinyl and pyrazinyl.

The term “heteroarylalkyl” refers to a group -alkyl-neteroaryl wherein alkyl and neteroaryl are as herein defined.

The term “heterocyclyl” or “heterocycle” refers to non-aromatic, fully saturated or partially unsaturated cyclic groups (for example, 3 to 7 member monocyclic, 7 to 11 member bicyclic, or containing a total of 3 to 10 ring atoms) which have at least one heteroatom in at least one carbon atom-containing ring. Preferably the heterocyclyl is a 5- or 6-membered heterocyclyl. Each ring of the heterocyclic, group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms. Non limiting exemplary heterocyclic groups include piperidinyl, piperazinyl, azetidinyl, azocanyl, diazepanyl, diazocanyl, morpholin-4-yl, oxazepanyl, pyrrolidinyl, thiomorpholin-4-yl, tetrahydrofuranyl, tetrahydropyranyl, aziridinyl, oxiranyl, thiirarnyl, 2-imidazolinyl, pyrazolidinyl imidazolidinyl, isoxazolinyl, oxazolidinyl, isoxazolidinyl, thiazolidinyl, isothiazolidinyl, succinimidyl, 3H-indolyl, indolinyl, isoindolinyl, 2H-pyrrolyl, 1-pyrrolinyl, 2-pyrrolinyl, 3-pyrrolinyl, 4H-quinolizinyl, 2-oxopiperazinyl, homopiperazinyl, 2-pyrazolinyl, 3-pyrazolinyl, tetrahydro-2H-pyranyl, 2H-pyranyl, 3,4-dihydro-2H-pyranyl, oxetanyl, thietanyl, 3-dioxolanyl, 1,4-dioxanyl, 2,5-dioximidazolidinyl, 2-oxopiperidinyl, 2-oxopyrrolodinyl, indolinyl, tetrahydrothiophenyl, tetrahydroquinolinyl, tetrahydroisoquinolinyl, 1-oxido-1-thiomorpholin-4-yl, 1-dioxido-1-thiomorpholin-4-yl, 1,3-dioxolanyl, 1,4-oxathanyl, 1,4-dithianyl, 1,3,5-trioxanyl, 1H-pyrrolizinyl, tetrahydro-1,1-dioxothiohenyl, N-formylpiperazinyl, dihydrotriazolopyrazine, dihydroimidazopyrazine, hexahydropyrrolopyrrole, hexahydropyrrolopyrazine.

The term “heterocyclylalkyl” refers to a group -alkyl-heterocyclyl wherein alkyl and heterocyclyl are as herein defined.

The term “heterocyclylalkylaminocarbonyl” refers to a group —(C═O)—NH-alkyl-heterocyclyl wherein alkyl and heterocyclyl are as herein defined.

The term “(heterocyclyl)(alkyl)aminoalkyl” refers to a group -alkyl-NR¹R² wherein R¹ is an alkyl group and R² is a heterocyclyl group, wherein alkyl and heterocyclyl are as herein defined.

The term “hetrocyclylalkyloxyalkyl” refers to a group -alkyl-O-alkyl-heterocyclyl wherein alkyl and heterocyclyl are as herein defined.

The term “heterocyclylcarbonyl” refers to a group —(C═O)-heterocyclyl wherein heterocyclyl is as herein defined.

The term “heterocyclyloxy” to a group —O-heterocyclyl wherein heterocyclyl is as herein defined.

The term “heterocyclylsulfonyl” refers to a group —SO₂-heterocyclyl wherein heterocyclyl is as herein defined.

The term “hydroxy” or “hydroxyl” refers to a group

The term “hydroxyalkyl” refers to a group -alkyl-OH wherein alkyl is as herein defined.

The term “hydroxyalkylaminoalkyl” refers to a group -alkyl-NH-alkyl-OH wherein alkyl is as herein defined.

The term “hydroxycarbonyl” refers to a group —C(═O)—OH wherein carbonyl is as herein defined. In other words, “hydroxycarbonyl” corresponds to a carboxylic acid group.

The term “oxo” refers to a ═O substituent.

The term “sulfonyl amino” refers to a group —NH—SO₂.

The term “intermediate” or “intermediate compound” refers to a compound which is produced in the course of a chemical synthesis, which is not itself the final product, but is used in further reactions which produce the final product. There may be many different intermediate compounds between the starting material and end product in the course of a complex synthesis.

The term “about”, preceding a figure encompasses plus or minus 10%, or less, of the value of said figure. It is to be understood that the value to which the term “about” refers is itself also specifically, and preferably, disclosed.

The term “administration”, or a variant thereof (e.g. “administering”), means providing the active agent or active ingredient, alone or as part of a pharmaceutically acceptable composition, to the patient in whom/which the condition, symptom, or disease is to be treated or prevented.

The term “antagonist” refers to a natural or synthetic compound which binds to the protein and blocks the biological activation of the protein, and thereby the action of the said protein. The protein may be a receptor, i.e. a protein molecule that receives chemical signals from outside a cell. Consequently, “an adenosine receptor antagonist” includes any chemical entity that, upon administration to a patient, results in inhibition or down-regulation of a biological activity associated with activation of an adenosine receptor in the patient, including any of the downstream biological effects otherwise resulting from the binding to an adenosine receptor of its natural ligand. Such adenosine receptor antagonists include any agent that can block activation of an adenosine receptor or any of the downstream biological effects of an adenosine receptor activation.

The term “inhibitor” refers to a natural or synthetic compound that has a biological effect to inhibit or significantly reduce or down-regulate the expression of a gene and/or a protein or that has a biological effect to inhibit or significantly reduce the biological activity of a protein.

As used herein, the term “combination” preferably means a combined occurrence of the two or more therapeutic agents. In some embodiments, a combination of the present disclosure may occur either as one composition, comprising all the components in one and the same mixture (e.g. a pharmaceutical composition), or may occur as a kit of parts, wherein the different components form different parts of such a kit of parts. Administration of each compound of a combination of the present disclosure may occur either simultaneously or timely staggered, with similar or different timing of administration (i.e. similar or different numbers of administration of each component), either at the same site of administration or at different sites of administration, under similar of different dosage form.

The term “chemotherapy” refers to a type of cancer treatment that uses one or more anti-cancer drugs (chemotherapeutic agents) as part of a standardized chemotherapy regimen. Chemotherapy may be given with a curative intent or it may aim to prolong life or to reduce symptoms. Chemotherapeutic agents are for example selected from anticancer alkylating agents, anticancer antimetabolites, anticancer antibiotics, plant-derived anticancer agents, anticancer platinum coordination compounds and any combination thereof.

The term “hormone therapy” refers to the use of hormones in medical treatment. In one embodiment, the hormone therapy is oncologic hormone therapy.

The term “human” refers to a subject of both genders and at any stage of development (i.e. neonate, infant, juvenile, adolescent, adult).

The term “subject” and “patient” are used interchangeably and refer to a mammal, more preferably a human, who/which is awaiting the receipt of, or is receiving medical care or is/will be the object of a medical procedure. In some embodiments, a subject has previously received treatment with a A_{2A} R antagonist. The term “subject” refers to a mammal, preferably a human. In one embodiment, the subject is diagnosed with a cancer. In one embodiment, the subject is a patient, preferably a human patient, who/which is awaiting the receipt of, or is receiving, medical care or was/is/will be the subject of a medical procedure or is monitored for the development or progression of a disease, such as a cancer. In one embodiment, the subject is a human patient who is treated and/or monitored for the development or progression of a cancer. In one embodiment, the subject is a male. In another embodiment, the subject is a female. In one embodiment, the subject is an adult. In another embodiment, the subject is a child.

The term “immunotherapy” refers to a therapy aiming at inducing and/or enhancing an immune response towards a specific target, for example towards cancer cells. Immunotherapy may involve the use of checkpoint inhibitors, checkpoint agonists (also called T-cell agonists), IDO inhibitors, PI3K inhibitors, adenosine receptor inhibitors, adenosine-producing enzymes inhibitors, adoptive transfer, therapeutic vaccines, and combinations thereof.

The expression “pharmaceutically acceptable” refers to the ingredients of a pharmaceutical composition are compatible with each other and not deleterious to the subject to which it is administered.

The expression “pharmaceutically acceptable carrier, diluent, excipient and/or adjuvant” refers to a substance that does not produce an adverse, allergic or other untoward reaction when administered to an animal, preferably a human. It includes any and all inactive substance such as for example solvents, cosolvents, antioxidants, surfactants, stabilizing agents, emulsifying agents, buffering agents, pH modifying agents, preserving agents (or preservating agents), antibacterial and antifungal agents, isotonifiers, granulating agents or binders, lubricants, disintegrants, glidants, diluents or fillers, adsorbents, dispersing agents, suspending agents, coating agents, bulking agents, release agents, absorption delaying agents, sweetening agents, flavoring agents and the like. For human administration, preparations should meet sterility, pyrogenicity, general safety and purity standards as required by regulatory offices, such as, e.g., FDA Office or EMA.

The terms “prevent”, “preventing” and “prevention”, as used herein, refer to a method of delaying or precluding the onset of a condition or disease and/or its attendant symptoms, barring a patient from acquiring a condition or disease, or reducing a patient's risk of acquiring a condition or disease.

The term “prodrug” as used herein means the pharmacologically acceptable derivatives of compounds of Formula (1), such as for example esters or amides, whose in vivo biotransformation product generates the biologically active drug. Prodrugs are generally characterized by increased bio-availability and are readily metabolized into biologically active compounds in vivo.

The term “radiation therapy” refers to a method of treatment of cancer employing various radiations such as X-ray, gamma ray, neutron ray, electron beam, proton beam and radiation sources. It is used as part of cancer treatment to control or kill malignant cells. Radiation therapy may be curative in a number of types of cancer if they are localized to one area of the body. It may also be used as part of adjuvant therapy, to prevent tumor recurrence after surgery to remove a primary malignant tumor. The three main divisions of radiation therapy are: external beam radiation therapy (EBRT or XRT); brachytherapy or sealed source radiation therapy; and systemic radioisotope therapy (RIT) or unsealed source radiotherapy.

The terms “therapeutically effective amount” or “effective amount” or “therapeutically effective dose” refer to the amount or dose of active ingredient that is aimed at, without causing significant negative or adverse side effects to the subject, (1) delaying or preventing the onset of a cancer in the subject; (2) reducing the severity or incidence of a cancer; (3) slowing down or stopping the progression, aggravation, or deterioration of one or more symptoms of a cancer affecting the subject; (4) bringing about ameliorations of the symptoms of a cancer affecting the subiect or (5) curing a cancer affecting the subject. A therapeutically effective amount may be administered prior to the onset of a cancer for a prophylactic or preventive action. Alternatively, or additionally, a therapeutically effective amount may be administered after initiation of a cancer for a therapeutic action.

The terms “treating” or “treatment” refer to therapeutic treatment; wherein the object is to prevent or slow down the targeted pathologic condition or disease. A subject or mammal is successfully “treated” for a disease or affection or condition if, after receiving the treatment according to the present disclosure, the subject or mammal shows observable and/or measurable reduction in or absence of one or more of the following: reduction of the number of cancer cells; and/or relief to some extent, for one or more of the symptoms associated with the specific disease or condition; reduced morbidity and mortality, and improvement in quality of life issues. The above parameters for assessing successful treatment and improvement in the disease are readily measurable by routine procedures familiar to a physician.

The term “stem cell transplant” refers to a procedure in which a patient receives healthy blood-forming cells (stem cells) to replace their own that have been destroyed by disease or by the radiation or high doses of anticancer drugs that are given as part of the procedure. The healthy stem cells may come from the blood or bone marrow of the patient, from a donor, or from the umbilical cord blood of a newborn baby. A stem cell transplant may be autologous (using a patient's own stem cells that were collected and saved before treatment), allogeneic (using stem cells donated by someone Who is not an identical twin), or syngeneic (using stem cells donated by an identical twin).

### DETAILED DESCRIPTION

We have demonstrated that T cell proliferation and cytokine production is suppressed in the presence of high adenosine concentrations as found in the tumor microenvironment. We have also demonstrated that A_{2A} receptor antagonists can restore T cell activity in tumors with high adenosine concentrations (see. e.g. PCT Publication WO2018/178338 “2-Oxo-Thiazole Derivatives as A_{2A} inhibitors and Compounds for Use in the Treatment of Cancers,” US Publication 2020/0102319 “Non Brain Penetrant A2A Inhibitors and Methods for Use in the Treatment of Cancers,” and PCT Publication WO2020/065036 “Thiocarbainate Derivatives as A_{2A} Inhibitors, Pharmaceutical Composition Thereof and Combinations with Anticancer Agents,” each of which is incorporated by reference in its entirety). In particular, the present disclosure shows among other things that using A_{2A} receptor as a hiomarker is associated with clinical benefit.

### Detection of A_{2A}

In some embodiments, the present disclosure provides a method to determine if a subject has an elevated level of A_{2A} receptor expression comprising: (a) detecting the level of A_{2A} receptor expression in a sample from the subject using an in vitro assay and (b) comparing the level of the A_{2A} receptor expression to a suitable reference level of A_{2A} receptor expression. In some embodiments, a subject with elevated A_{2A} receptor expression is administered a compound or a combination of compounds effective for treatment of a patient having an elevated level of A_{2A} receptor expression. In some embodiments, a subject with elevated A_{2A}R expression is selected for treatment with a combination of compounds effective for treatment of a patient having an elevated level of A_{2A}R expression. In some embodiments, the compound or combination of compounds include an adenosine receptor antagonist, as further defined below. In some embodiments, the adenosine receptor antagonist is an antagonist of A₁ receptor, A_{2A} receptor, A_{2B} receptor, A₃ receptor or of a combination thereof; preferably the adenosine receptor antagonist is an antagonist of A_{2A} receptor, A_{2B} receptor or of a combination thereof; more preferably the adenosine receptor antagonist is an A_{2A} receptor antagonist.

In some embodiments, the present disclosure provides a method to determine if a subject has an elevated density of A_{2A}R⁺ cells comprising: (a) detecting the density of A_{2A}R⁺ cells in a sample from the subject using an in vitro assay and (b) comparing the density of A_{2A}R⁺ cells to a suitable reference level of density of A_{2A}R⁺ cells. In some embodiments, a subject with a tumor with elevated density of A_{2A}R⁺ cells is administered a compound or a combination of compounds effective for treatment of a patient with a tumor having an elevated density of A_{2A}R⁺ cells. In some embodiments, a subject with a tumor with elevated density of A_{2A}R⁺ cells is selected for treatment with a compound or a combination of compounds effective for treatment of a patient with a tumor having an elevated density of A_{2A}R⁺ cells. In some embodiments, the compound or combination of compounds include an adenosine receptor antagonist, as further defined below. In some embodiments, the adenosine receptor antagonist is an antagonist of A₁ receptor, A_{2A} receptor, A_{2B} receptor, A₃ receptor or of a combination thereof; preferably the adenosine receptor antagonist is an antagonist of A_{2A} receptor, A_{2B} receptor or of a combination thereof; more preferably the adenosine receptor antagonist is an A_{2A} receptor antagonist.

In some embodiments, A_{2A}R⁺ cells are lymphocytes. In some embodiments, A_{2A}R⁺ cells are tumor-infiltrating lymphocytes.

In some embodiments, A_{2A} receptor expression or density of A_{2A}R⁺ cells may be determined using a suitable in vitro assay. In some embodiments, A_{2A} receptor expression or density of A_{2A}R⁺ cells may be determined by measuring the levels, amount or concentration of A_{2A}R at the RNA or protein levels. In some embodiments, A_{2A} expression or density of A_{2A}R⁺ cells may be determined by measuring a RNA or protein level in a sample. in vitro assays for measuring the level, amount or concentration of A_{2A}R at the RNA level include, without limitation, reverse-transcriptase quantitative polymerase chain reaction (RT-qPCR), in situ hybridization (ISH), DNA microarrays, Nanostring technology, and the like. in vitro assays for measuring the level, amount or concentration of A_{2A}R at the protein level include, without limitation, immunohistochemistry (IHC), fluorescent IHC, flow cytometry, Western blotting, enzyme-linked immunosorbent assay (ELISA), and the like.

In some embodiments, A_{2A} receptor expression or density of A_{2A}R⁺ cells in a tumor is compared to a control, i.e., a suitable reference standard.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present disclosure. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods of the disclosure are not limited to use of a specific cut-point in comparing the level of expression product in the test sample to the control.

In some embodiments, a suitable control or reference standard is A_{2A} receptor expression level or density of A_{2A}R⁺ cells in a subject not affected and/or diagnosed with cancer. In some embodiments, a suitable reference standard is the mean A_{2A}R expression level or mean density of A_{2A}R⁺ cells in a population of subjects not affected and/or diagnosed with cancer. In some embodiments, a suitable reference standard is A_{2A} receptor expression level or density of A_{2A} R⁺ cells of a sample from the subject themselves. In some embodiments, a suitable reference standard is A_{2A} receptor expression level or density of A_{2A}R⁺ cells in a noncancerous cellular sample adjacent to a tumor from the subject themselves.

In some embodiments, the present disclosure includes determining a level of A_{2A} receptor expression or density of A_{2A}R⁺ cells in a tumor in a subject comprising obtaining or having obtained a biological sample from the subject; and performing an assay on the biological sample to determine if the tumor has a elevated level of A_{2A} receptor expression or density of A_{2A}R⁺ cells.

In some embodiments, the methods of determining a level of A_{2A}R expression or density of A_{2A}R⁺ cells disclosed herein are in vitro method. In other words, the methods are non-invasive and do not include a step of taking a sample from the subject. In some embodiments, the methods are performed on a sample previously obtained from the subject.

In some embodiments, the sample is a bodily fluid. In some embodiments, the sample is a bodily tissue. In some embodiments, the sample is a tumor tissue sample. In some embodiments, the tumor tissue sample comprises tumor cells. In some embodiments, the tumor tissue sample further comprises tumor infiltrating immune cells. In some embodiments, the tumor tissue sample does not comprise tumor infiltrating immune cells.

In some embodiments, the level of A_{2A}R expression is considered as “elevated” or “increased” or “higher” when said level is at least 5%, 10%, 15%, 20%, 25%, 30%, 35%, 40%, 45%, 50%, 55%, 60%, 65%, 70%, 75%, 80%, 85%, 90%, 95%, 100% or more higher than the level of A_{2A}R expression is a control subject or population, e.g., in a subject or a population of subjects not affected and/or diagnosed with cancer, or in a sample such as a non-cancerous sample from the subject themselves.

In some embodiments, the level of A_{2A}R expression is considered as “elevated” or “increased” or “higher” When said level is above about 3.5, 3.6, 3.7, 3.8, 3.9, 4.0, 4.1, 4.2, 4.3, or 4.4. In some embodiments, the level of A_{2A}R expression is considered as “elevated” or “increased” or “higher” when said level is above about 3.95. in some embodiments, the level of A_{2A}R expression is considered as “elevated” or “increased” or “higher” when the A_{2A}R log2 expression value relative to the reference value is greater than about 3.5-4.5. In some embodiments, the level of A_{2A}R expression is considered as “elevated” or “increased” or “higher” when the A_{2A}R log2 expression value relative to the reference value is greater than about 3.7-4.3. In some embodiments, the level of A_{2A}R expression is considered as “elevated” or “increased” or “higher” when the A_{2A}R log2 expression value relative to the reference value is greater than about 3.9-4.0. In some embodiments, the level of A_{2A}R expression is considered as “elevated” or “increased” or “higher” when the A_{2A}R log2 expression value relative to the reference value is greater than about 3.5, 3.6, 3.7. 3.8, 3.9, 4.0, 4.1, 4.2, 4.3, or 4.4. In some embodiments, the level of A_{2A}R expression is considered as “elevated” or “increased” or “higher” when the A_{2A}R log2 expression value relative to the reference value is greater than about 3.95.

In some embodiments, the density of A_{2A}R⁺ cells is considered as “elevated” or “increased” or “higher” when said density is at least 5%, 10%, 15%, 20%, 25%, 30%, 35%, 40%, 45%, 50%, 55%, 60%, 65%, 70%, 75%, 60%, 85%, 90%, 95%, 100% or more higher than the density of A_{2A}R⁺ cells is a control subject or population, e.g., in a subject or a population of subjects not affected and/or diagnosed with cancer, or in a sample such as a non-cancerous sample from the subject themselves.

In some embodiments, the density of A_{2A}R⁺ cells is considered as “elevated” or “increased” or “higher” when said density is equal or above about 13, 14, 15, 16, 17, 18, 19, 20, 21, 22, 23 or more A_{2A}R⁺ cells/mm², preferably equal or above about 18 A_{2A}R⁺ cells/mm². In some embodiments, the density of A_{2A}R⁺ cells is considered as “elevated” or “increased” or “higher” when said density is equal or above about 13-23 cells/mm². In some embodiments, the density of A_{2A}R⁺ cells is considered as “elevated” or “increased” or “higher” when said density is equal or above about 15-21 cells/mm². In some embodiments, the density of A_{2A}R⁺ cells is considered as “elevated” or “increased” or “higher” When said density is equal or above about 17-19 cells/mm². In some embodiments, the density of A_{2A}R⁺ cells may be determined in a 4-to-5 μm-thick section of a bodily tissue, in particular of a tumor tissue.

### Adenosine Receptor Antagonists and Methods of use Thereof

As defined above, “adenosine receptor antagonist” refers to a compound that, upon administration to a patient, results in inhibition or down-regulation of a biological activity associated with activation of an adenosine receptor in the patient, including any of the downstream biological effects otherwise resulting from the binding to an adenosine receptor of its natural ligand. Such adenosine receptor antagonists include any agent that can block activation of an adenosine receptor or any of the downstream biological effects of an adenosine receptor activation.

Adenosine receptors (or P1 receptors) are a class of purinergic G protein-coupled receptors with adenosine as endogenous ligand. There are four known types of adenosine receptors in humans: A₁, A_{2A}, A_{2B} and A₃; each is encoded by a different gene (ADOARA1, ADORA2A, ADORA2B, and ADORA3 respectively).

In one embodiment, an adenosine receptor antagonist is an antagonist of A₁ receptor, A_{2A} receptor, A_{2B} receptor, A₃ receptor or of a combination thereof.

In one embodiment, an adenosine receptor antagonist is an antagonist of A_{2A} receptor, A_{2B} receptor or of a combination thereof. In one embodiment, an adenosine receptor antagonist is an A_{2A} or A_{2B} receptor antagonist.

In one embodiment, an adenosine receptor antagonist is an antagonist of A_{2A} receptor (A_{2A}R antagonist). In one embodiment, the adenosine receptor antagonist is an antagonist of A_{2B} receptor (A_{2B}R antagonist).

In one embodiment, an adenosine receptor antagonist is an antagonist that is selective of A_{2A} receptor with respect to other adenosine receptors. In one embodiment, the adenosine receptor antagonist is an antagonist that is selective of A_{2A} receptor with respect to A_{2B} receptor.

In one embodiment, an adenosine receptor antagonist is an antagonist that is selective of A_{2B} receptor with respect to other adenosine receptors. In one embodiment, an adenosine receptor antagonist is an antagonist that is selective of A_{2B} receptor with respect to A_{2A} receptor.

### A_{2A} Receptor Antagonists

An “A_{2A}R antagonist” refers to a compound that, upon administration to a subject, results in inhibition or down-regulation of a biological activity associated with activation of A_{2A} receptor in the patient, including any of the downstream biological effects otherwise resulting from the binding to A_{2A} receptor of its natural ligand. Such A_{2A}R antagonists include any agent that can block activation of A_{2A} receptor or any of the downstream biological effects of A_{2A} receptor activation.

In some embodiments, an A_{2A}R antagonist includes, but is not limited to, Preladenant (SCH-420,814), Vipadenant (BIIB-014), Tozadenant (SYX-115), ATL-444, Istradefylline (KW-6002), MSX-3, SCH-58261, SCH-412,348 SCH-442,416, SCH-1535, Caffeine, VER-6623, VER-6947, VER-7835, ZM-241,385, theophylline. In some embodiments, an A_{2A}R antagonist includes, but is not limited to, compounds disclosed in WO2018/178338, WO2011/121418, WO2009/156737, WO2011/095626 or WO2018/136700, the content of which is herein incorporated by reference.

In one embodiment, an A_{2A}R antagonist is a thiocarbamate disclosed in WO2018/178338. More preferably an A_{2A}R antagonist is a compound of formula (II):

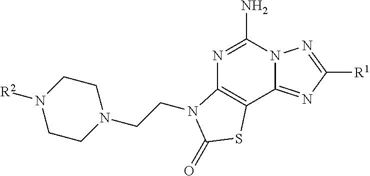

or a pharmaceutically acceptable salt or solvate thereof,
   wherein
R¹ represents 5- or 6-membered heteroaryl or 5- or 6-membered aryl, wherein heteroaryl or aryl groups are optionally substituted by one or more substituent selected from C1-C6 alkyl (preferably methyl) and halo (preferably fluoro or chloro); preferably R¹ represents 5-membered heteroaryl; more preferably R¹ represents furyl;
R² represents 6-membered aryl or 6-membered heteroaryl, wherein heteroaryl or aryl groups are optionally substituted by one or more substituent selected from halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkyl sulfonyl, aminosulfonyl, heterocyclylsulfonyl, alkylsulfonimidoyl, carbonylamino, sulfonylamino and alkylsulfonealkyl;
   said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl alkylsulfonyl and alkylsulfonealkyl;
   or the heteroaryl or aryl groups are optionally substituted with two substituents that form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkyl sulfoxidealkyl, alkylsulfonyl and alkylsulfonealkyl.

In one embodiment, an A_{2A}R antagonist is a compound of Formula (11a):

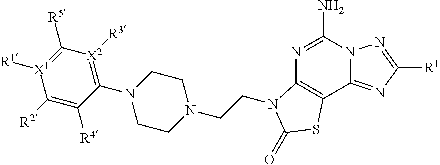

or a pharmaceutically acceptable salt or solvate thereof,
   wherein:
R¹ represents 5- or 6-membered heteroaryl or 5- or 6-membered aryl, wherein heteroaryl or aryl groups are optionally substituted by one or more substituent selected from C1-C6 alkyl (preferably methyl) and halo (preferably fluoro or chloro); preferably R¹ represents 5-membered heteroaryl; more preferably R¹ represents furyl;
X¹ and X² represent each independently C or N;
R^{1′} is absent when X¹ is N; or when X¹ is C, R^{1′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkyl sulfonyl, aminosulfonyl, heterocyclylsulfonyl, alkylsulfonimidoyl, carbonylamino, sulfonylamino or alkylsulfonealkyl;
   said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkyl sulfoxidealkyl, alkylsulfonyl and alkylsulfonealkyl;
R^{2′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkyl sulfonyl, aminosulfonyl, heterocyclylsulfonyl, alkylsulfonimidoyl, carbonylamino, sulfonylamino, or alkylsulfonealkyl;
   said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkyl sulfoxidealkyl, alkylsulfonyl and alkylsulfonealkyl;
or R^{1′} and R^{2′} form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkyl sulfoxidealkyl, alkyl sulfonyl and alkylsulfonealkyl;
R^{3′} is absent when X² is N; or when X² is C, R^{3′} represents H or halo, preferably H or F;
R^{4′} represents H or halo, preferably H or F; and
R^{5′} represents H or halo, preferably H or F.

In some embodiments, an A_{2A}R antagonist is a compound of Formula (11a-1):

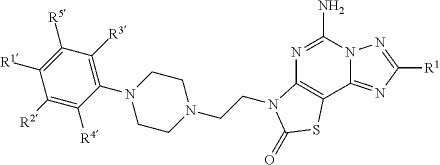

or a pharmaceutically acceptable salt or solvate thereof, wherein R¹, R^{1′}, R^{2′}, R^{3′}, R^{4′} and R^{5′} are as defined in Formula (IIa).

In some embodiments, an A_{2A}R antagonist is a compound of Formula (IIa-1a):

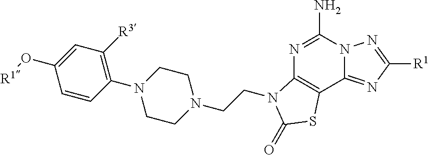

or a pharmaceutically acceptable salt or solvate thereof,
   wherein:
R¹ and R^{3′} are as defined in Formula (Ia); and
R^{1″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkyl sulfoxidealkyl, alkylsulfonyl and alkylsulfonealkyl.

In one embodiment, a A_{2A}R antagonist is a compound of Formula (IIa-1b):

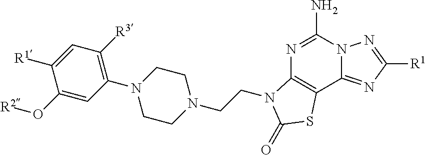

or a pharmaceutically acceptable salt or solvate thereof,
   wherein:
R¹ and R^{3′} are as defined in Formula (IIa);
R^{1′} represents H or halo, preferably H or F; and
R^{2″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkyl sulfoxidealkyl, alkylsulfonyl and alkylsulfonealkyl.

In one embodiment, an A_{2A}R antagonist is a compound of Formula (IIa-1c) or (IIa-1d):

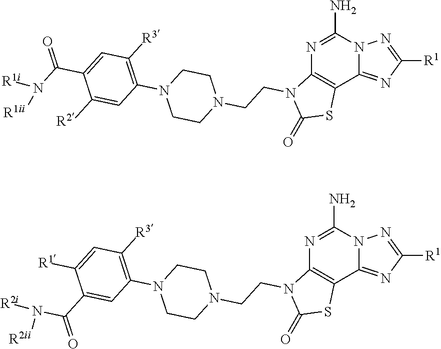

or a pharmaceutically acceptable salt or solvate thereof,
   wherein:
R¹ and R^{3′} are as defined in Formula (Ia);
R^{1′} represents H or halo, preferably H or F;
R^{2′} represents H or halo, preferably H or F;
R¹ⁱ and R¹ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl; and
R²ⁱ and R²ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl.

In some embodiments, an A_{2A}R antagonist is a compound of Formulae (IIa-2) or (IIa-3):

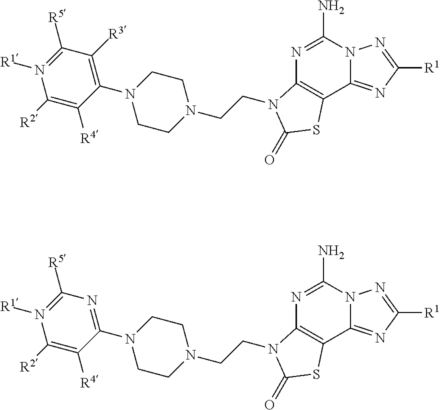

or a pharmaceutically acceptable salt or solvate thereof,
   wherein R¹, R^{2′}, R^{3′}, R^{4′} and R^{5′} are as defined in Formula (IIa).

In some embodiments an A_{2A}R antagonist is a compound selected from the group consisting of:

3-(2-(4-(4-((1H-1,2,3-triazolo-4yl)methoxy-2fluorophenyl)piperazine-1-yl)ethyl)-5-amino-(8-(furan-2-yl)thiazolo[5,4-e][1,2,4]triazolo[1,5-c]pyrimidine-2(3H)-one;
5-((4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)methyl)-1,3,4-oxadiazol-2(3H)-one;
5-amino-3-(2-(4-(3-fluoropyridin-4-yl)piperazin-1-yl)ethyl)-8-(furan-2-yl)thiazolo[5,4-e][1,2,4]triazolo[1,5-c]pyrimidin-2(3H)-one;
2-(5-(4-(2-(5-amino-8-(furan-2-yl)-2-oxothiazolo[5,4-e][1,2,4]triazolo [1,5-c] pyrimidin-3(2H)-yl)ethyl)piperazin-1-yl)-2,4-difluorophenoxy)acetamide;
(S)-5-amino-3-(2-(4-(2-fluoro-4-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2-fluoro-4-(2-(methylsulfinyl)ethoxy)phenyl)-piperazin-1-yl)ethyl)-8-(furan-2-yl)thiazolo[5,4-e][1,2,4]triazolo[1,5-c]pyrimidin-2(3H)-one;
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(+)-5-amino-3-(2-(4-(2,4-difluoro-5-(2-(methylsulfinyl)ethoxy)phenyl)piperazin-1-ypethyl)-8-(furan-2-yl)thiazolo[5,4-e][1,2,4]triazolo[1,5-c]pyrimidin-2(3H)-one;
(−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-8-(furan-2-yl)-3-(2-(4-(4-(2-hydroxyethoxy)phenyl)piperazin-1-yl)ethyl)thiazolo[5,4-e][1,2,4]triazolo[1,5-c]pyrimidin-2(3H)-one;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)phenoxy)acetic acid;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)phenoxy)acetamide;
5-amino-3-(2-(4-(4-(2,3-dihydroxypropoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(4-(2-aminoethoxy)phenyl)piperazin-1-yl)ethyl)-8-(furan-2-yl)thiazolo[5,4-e][1,2,4]triazolo[1,5-c]pyrimidin-2(3H)-one;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-methylbenzamide;
5-amino-8-(furan-2-yl)-3-(2-(4-(4-(2-morpholinoethoxy)phenyl)piperazin-1-yl)ethyl)thiazolo[5,4-e][1,2,4]triazolo[1,5-c]pyrimidin-2(3H)-one;
5-amino-3-(2-(4-(4-(2-(dimethylamino)ethoxy)phenyl)piperazin-1-yl)ethyl)-8-(furan-2-yl)thiazolo[5,4-e][1,2,4]triazolo[1,5-c]pyrimidin-2(3H)-one;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzenesulfonamide;
4-(4-(2-(5-amino-8-(furan-2-yl)-2-oxothiazolo[5,4-e][1,2,4]triazolo[1,5-c]pyrimidin-3(2H)-yl)ethyl) piperazin-1-yl)-N-methylbenzenesulfonamide;
5-amino-8-(furan-2-yl)-3-(2-(4-(4-(methylsulfonyl)phenyl)piperazin-1-yl)ethyl)thiazolo[5,4-e][1,2,4]triazolo[1,5-c]pyrimidin-2(3H)-one;
5-amino-8-(furan-2-yl)-3-(2-(4-(4-(methylsulfinyl)phenyl)piperazin-1-yl)ethyl)thiazolo[5,4-e][1,2,4]triazolo[1,5-c]pyrimidin-2(3H)-one;
3-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzamide;
5-amino-8-(furan-2-yl)-3-(2-(4-(3-(2-hydroxyethoxy)phenyl)piperazin-1-yl)ethyl)thiazolo[5,4-e][1,2,4]triazolo[1,5-c]pyrimidin-2(3H)-one;
5-amino-3-(2-(4-(2-fluoro-4-(2-oxo-2-(piperazin-1-yl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(piperidin-4-ylmethoxy) phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(piperazine-1-carbonyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(2-(piperazin-1-yl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(piperazin-1-ylsulfonyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(methylsulfonyl)phenyl)piperazin-1-yl)ethyl)-8-(furan-2-yl)thiazolo[5,4-e][1,2,4]triazolo[1,5-c]pyrimidin-2(3H)-one;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-aminoethyl)-3-fluorobenzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylamino)ethyl) benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-3-fluorobenzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-hydroxyethyl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-3-fluorobenzamide;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)acetic acid;
2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3,5-difluorophenoxy) acetic acid;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid;
(S)-2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-2-methylpropanoic acid;
3-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenyl)propanoic acid;
4-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)butanoic acid;
2-(3-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,6-difluorophenoxy) acetic acid;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) acetic acid;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorobenzoic acid;
2-((2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)amino)acetamide;
2-((2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3 -fluorophenoxy)ethyl)(methyl)amino)acetamide;
5-amino-3-(2-(4-(2-fluoro-4-(piperidin-4-yloxy)phenyl)piperazin-1-yl) ethyl)-8-(furan-2-yl)thiazolo[5,4-e][1,2,4]triazolo[1,5-c]pyrimidin-2(3H)-one;
5-amino-3-(2-(4-(2-fluoro-4-(pyrrolidin-3-yloxy)phenyl)piperazin-1-yl)ethyl)-8-(furan-2-yl)thiazolo[5,4-e][1,2,4]triazolo[1,5-c]pyrimidin-2(3H)-one;
3-(2-(4-(4-((1H-1,2,4-triazol-3-yl)methoxy)-2-fluorophenyl)piperazin-1-yl)ethyl)-5-amino-8-(furan-2-yl)thiazolo[5,4-e][1,2,4]triazolo[1,5-c]pyrimidin-2(3H)-one;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-(methylamino)ethyl) acetamide;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-(dimethylamino)ethyl)acetamide;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-aminoethyl)acetamide;
(R)-2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)acetamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-methyl-N-(2-(methylamino)ethyl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-3-fluoro-N-methylbenzamide;
(R)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(1-(dimethylamino) propan-2-yl)-3-fluorobenzamide;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-methyl-N-(2-(methylamino)ethyl)acetamide;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-2-methylpropanoic acid;
(S)-2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)propanoic acid;
(R)-2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)propanoic acid;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-(methylamino)ethyl)acetamide;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-(dimethylamino)ethyl)acetamide;
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-2,4-difluoro-N-methylbenzamide;
4-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)butanoic acid;
3-(2-(4-(54(1H-tetrazol-5-yl)methoxy)-2,4-difluorophenyl)piperazin-1-yl)ethyl)-5-amino-8-(furan-2-yl)thiazolo[5,4-e][1,2,4]triazolo[1,5-c]pyrimidin-2(3H)-one;
5-amino-3-(2-(4-(2-fluoro-4((1-methyl-1H-1,2,4-triazol-3-yl)methoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-((1-methyl-1H-1,2,4-triazol-3-yl)methoxy)phenyl)piperazin-1-yl)ethyl)-8-(furan-2-yl)thiazolo[5,4-e][1,2,4]triazolo[1,5-c]pyrimidin-2(3H)-one;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methyl(oxetan-3-yl)amino)ethyl) benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-((2-hydroxyethyl)amino)ethyl)benzamide;
2-amino-N-(2-(4-(4-(2-(5-amino-8-(furan-2-yl)-2-oxothiazolo[5,4-e][1,2,4]triazolo[1,5-c]pyrimidin-3(2H)-yl) ethyl)piperazin-1-yl)-3-fluorophenoxy)ethyl) acetamide;
(S)-2-amino-N-(2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)-3-methylbutanamide;
ethyl 2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) acetate;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) acetonitrile;
5-amino-8-(furan-2-yl)-3-(2-(4-(pyridin-4-yl) piperazin-1-yl)ethyl)thiazolo[5,4-e][1,2,4]triazolo[1,5-c]pyrimidin-2(3H)-one;
5-amino-8-(furan-2-yl)-3-(2-(4-(pyrimidin-4-yl)piperazin-1-yl)ethyl)thiazolo[5,4-e][1,2,4]triazolo[1,5-c]pyrimidin-2(3H)-one;
5-amino-3-(2-(4-(2,4-difluoro-5-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(6-fluoro-2-oxoindolin-5-yl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(S-methylsulfonimidoyl)phenyl)piperazin-1-yl)ethyl)-8-(furan-2-yl)thiazolo[5,4-e][1,2,4]triazolo[1,5-c]pyrimidin-2(3H)-one;
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-2,4-difluorobenzamide;
5-amino-3-(2-(4-(5-fluoro-2-methylpyridin-4-yl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-44(3R,4R)-4-hydroxytetrahydrofuran-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3S,4S)-4-hydroxytetrahydrofuran-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(2-hydroxy-2-methylpropoxy)phenyl)piperazin-1-yl)ethyl)-8-(furan-2-yl)thiazolo[5,4-e][1,2,4]triazolo[1,5-c]pyrimidin-2(3H)-one;
5-amino-3 -(2-(4-(2-fluoro-4-(2-hydroxypropan-2-yl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(3,3,3-trifluoro-2-hydroxypropoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-5-(2-hydroxyethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(morpholin-2-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(morpholin-3-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((3S,4 S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((3R,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((3S,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2,4-difluoro-54(2-oxopyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5 (R)-5-amino-3-(2-(4-(2,4-difluoro-54(2-oxopyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
2-(5-(4-(2-(5-amino-8-(furan-2-yl)-2-oxothiazolo[5,4-e][1,2,4]triazolo[1,5-c]pyrimidin-3 (2H)-yl)ethyl)piperazin-1-yl)-2,4-difluorophenoxy)-N-(2-morpholinoethyl)acetamide;
5-(4-(2-(5-amino-8-(furan-2-yl)-2-oxothiazolo[5,4-e][1,2,4]triazolo[1,5-c]pyrimidin-3 (2H)-yl)ethyl)piperazin-1-yl)-2,4-difluoro-N-(morpholin-3-ylmethyl)benzamide;
5-amino-3-(2-(4-(2-fluoro-4-(morpholin-3-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(morpholin-2-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3R,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3R,4 S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3S,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
2-(4-(4-(2-(5-amino-8-(furan-2-yl)-2-oxothiazolo [5,4-e] [1,2,4]triazolo[1,5-c]pyrimidin-3(2H)-yl)ethyl)piperazin-1-yl)-3 -fluorophenoxy)-N-(2-morpholinoethyl)acetamide;
4-(4-(2-(5-amino-8-(furan-2-yl)-2-oxothiazolo[5,4-e][1,2,4]triazolo[1,5-c]pyrimidin-3 (2H)-yl)ethyl)piperazin-1-yl)-3 -fluoro-N-(2-morpholinoethyl)benzamide;
4-(4-(2-(5-amino-8-(furan-2-yl)-2-oxothiazolo[5,4-e][1,2,4]triazolo[1,5-c]pyrimidin-3 (2H)-yl)ethyl)piperazin-1-yl)-3 -fluoro-N-(morpholin-3 -ylmethyl)benzamide;
5-amino-3-(2-(4-(4-(azetidin-3-yloxy)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((1s,4s)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((1r,4r)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide;
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methyl sulfinyl)ethyl)benzamide;
5-amino-3-(2-(4-(2,4-difluoro-5-(1-oxidothiomorpholine-4-carbonyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(1-oxidothiomorpholino)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((1 s,4 s)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((1r,4r)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;
(S)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide;
(R)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide;
5-amino-3-(2-(4-(2-fluoro-4-(1-oxidothiomorpholine-4-carbonyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(1-oxidothiomorpholino)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(5-(2,3-dihydroxypropoxy)-2,4-difluorophenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(5-(2,3-dihydroxypropoxy)-2,4-difluorophenyl)piperazin-1-yl)ethyl)-8-(furan-2-yl)thiazolo[5,4-e][1,2,4]triazolo[1,5-c]pyrimidin-2(3H)-one;
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-2,4-difluorobenzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-2,4-difluorobenzamide;
5-amino-3-(2-(4-(4-(azetidin-3-yloxy)-2-fluorophenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(5-(azetidin-3-yloxy)-2,4-difluorophenyl)piperazin-1-yl)ethyl)-8-(furan-2-yl)thiazolo[5,4-e][1,2,4]triazolo[1,5-c]pyrimidin-2(3H)-one; and
(S)-5-amino-3-(2-(4-(2,4-difluoro-5-(3-(methylsulfinyl)propoxy)phenyl)piperazin-1-yl)ethyl)-8-(furan-2-yl)thiazolo[5,4-e][1,2,4]triazolo[1,5-c]pyrimidin-2(3H)-one,
or a pharmaceutically acceptable salt thereof.

In some embodiments, an A_{2A}R antagonist is selected from the group consisting of:
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one,
   or a pharmaceutically acceptable salt thereof.

In some embodiments, an A ZA R antagonist is selected from the group consisting of:
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(S)-5-amino-3-(2-(4-(2,4-difluoro-5-(2-(methylsulfinyl)ethoxy)phenyl)piperazin-1-ypethyl)-8-(furan-2-yl)thiazolo[5,4-e][1,2,4]triazolo[1,5-c]pyrimidin-2(3H)-one,
   or a pharmaceutically acceptable salt thereof.

In some embodiments, an A_{2A}R antagonist is (+)-5-amino-3-(2-(4-(2,4-difluoro-5-(2-(methylsulfinypethoxy)phenyl)piperazin-1-yl)ethyl)-8-(furan-2-yl)thiazolo[5,4-e][1,2,4]triazolo[1,5-c]pyrimidin-2(3H)-one.

In some embodiments, an A_{2A}R antagonist is (−)-5-amino-3-(2-(4(2,4-difluoro-5.(2-5-(2-(methy;sulfinyl)ethoxy)phenyl)piperazin-1-yl)ethyl)-8-(furan-2-yl)thiazolo[5,4-e][1,2,4]triazolo[1,5-c]pyrimidin-2(3H)-one.

In another embodiment, an A_{2A}R antagonist is an A_{2A}R antagonist disclosed in WO2011/1214118, Especially, an A_{2A}R antagonist is the compound of example 1 of WO2011/121418, namely 5--bromo-2,6-di-(1H-pyrazol-1-yl)pyrirnidin-4-amine, also known as NIR178:

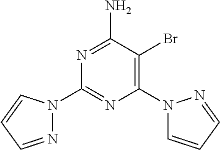 [SMILES: Nc1nc(-n2cccn2)nc(-n2cccn2)c1Br]

In another embodiment, an A_{2A}R antagonist is an A_{2A}R antagonist disclosed in WO2009/156737. Especially, an A_{2A}R antagonist is the compound of example 1S of WO2009/156737, namely (S)-7-(5-methylfuran-2-yl)-3-((6-(([tetrahydroluran-3-yl]oxy)methyl)pyridin-2-yl)methyl)-3H-[1,2,3]triazolo[4,5-d]pyrimidin-5-amine, also known as CPI-444:

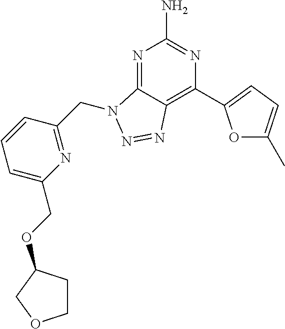 [SMILES: Cc1ccc(-c2nc(N)nc3c2nnn3Cc2cccc(CO[C@H]3CCOC3)n2)o1]

In another embodiment, an A_{2A}R antagonist is an A_{2A}R antagonist disclosed in WO2011/095626. Especially, the A_{2A}R antagonist is the compound (exiv) of WO2011/095626, namely 6-(2-chloro-6-methylpyridin-4-yl)-5-(4-fluorophenyl)-1,2,4-triazin-3-amine, also known as AZD4635:

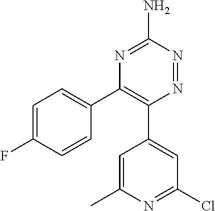 [SMILES: Cc1cc(-c2nnc(N)nc2-c2ccc(F)cc2)cc(Cl)n1]

In another embodiment, an A_{2A}R antagonist is an A_{2A}R antagonist disclosed in WO2018/136700. Especially, the A_{2A}R antagonist is the compound of example 1 of WO2018/136700, namely 3-(2-amino-6-(1((6-(2-hydroxypropan-2-yl)pyridin-2-yl)methyl)-1H-1,2,3-triazol-4-yl)pyrimidin-4-yl)-2-methylbenzonitrile, also known as AB928:

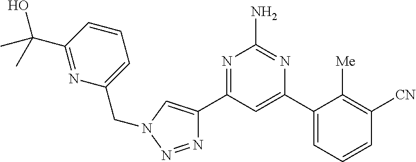 [SMILES: Cc1c(C#N)cccc1-c1cc(-c2cn(Cc3cccc(C(C)(C)O)n3)nn2)nc(N)n1]

In another embodiment, an A_{2A}R antagonist is Preladenant (SCH-420,814 namely 2-(2-furanyl)-7-(2-(4-(4-(2-methoxyethoxy)phenyl)-1-piperazinyl)-7H-pyrazolo(4,3-e)(1,2,4)triazolo(1,5-c)pyrimidine-5-amine:

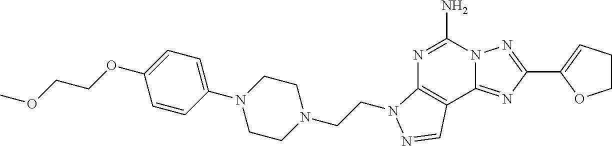 [SMILES: COCCOc1ccc(N2CCN(CCn3ncc4c3nc(N)n3nc(C5=CCCO5)nc43)CC2)cc1]

In another embodiment, an A_{2A}R antagonist is Vipadenant (BIIB-014), namely 3-(4-amino-3-methylbenzyl)-7-(2-furyl)-3H-(1,2,3)triazolo(4,5-d)pyrimidine-5amine:

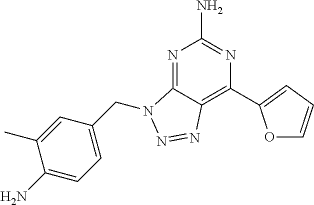 [SMILES: Cc1cc(Cn2nnc3c(-c4ccco4)nc(N)nc32)ccc1N]

In another embodiment, an A_{2A}R antagonist is Tozadenant (SYK-115), namely 4-hydroxy-N-(4-methoxy-7-morpholinobenzo[d]thiazol-2-yl)-4-methylpiperidine-1-carboxamide:

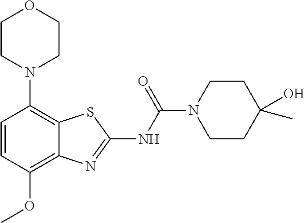 [SMILES: COc1ccc(N2CCOCC2)c2sc(NC(=O)N3CCC(C)(O)CC3)nc12]

In one embodiment, an adenosine receptor antagonist is selected from:

5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide.

In one embodiment, an adenosine receptor antagonist is 5-bromo-2,6-di-(1H-pyrazol-1-yl)pyrimidin-4-amine. In one embodiment, an adenosine receptor antagonist is (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine. In one embodiment, an adenosine receptor antagonist is 6-(2-chloro-6-methylpyridin-4-yl)-5-(4-fluorophenyl)-1,2,4-triazin-3-amine. In one embodiment, an adenosine receptor antagonist is 3-(2-amino-6-(1-((6-(2-bydroxypropan-2-yl)pyridin-2-yl)methyl)-1H-1,2,3-triazol-4-yl)pyrimidin-4-yl)-2-methylbenzonitrile.

A_{2A}R and A₃ Receptor Antagonists

An “A_{2B}R antagonist” refers to a compound that, upon administration to a patient, results in inhibition or down-regulation of a biological activity associated with activation of A_{2B} receptor in the patient, including any of the downstream biological effects otherwise resulting from the binding to A₃ receptor of its natural ligand. Such A_{2B}R antagonists include any agent that can block activation of A_{2B} receptor or any of the downstream biological effects of A_{2B} receptor activation.

Examples of A_{2B}R antagonists include: Vipadenant (BIIB-014), CVT-6883, MRS-1706, MRS-1754, PSB-603, PSB-0788, PSB-1115, OSIP-339,391, ATL-801, theophylline, or Caffeine.

Examples of inhibitors of A_{2B} receptor include ATL-801, CVT-6883 MRS-1706, MRS-1754, OSIP-339,391, PSB-603, PSB-0788 and PSB-1115.

Examples of inhibitors of receptor include KF-26777, MRS-545, MRS-1191, MRS-1220, MRS-1334, MRS-1523, MRS-3777, MRE-3005-F20, MRE-3008-F20, PSB-11, OT 7999, VUF-5574 and SSR161421.

### Methods

In some embodiments, the present disclosure includes a method of treating cancer characterized by increased A_{2A} receptor expression or density of ALAR: cells in a subject in need thereof, comprising administering to the subject a therapeutically effective amount of an adenosine receptor antagonist. In some embodiments, the present disclosure includes a method of treating cancer characterized by increased A_{2A} receptor expression or density of A_{2A}R⁺ cells in a tumor in a subject in need thereof, comprising administering to the subject a therapeutically effective amount of an adenosine receptor antagonist.

In some embodiments, the present disclosure includes an adenosine receptor antagonist, for use in the treatment of cancer in a subject in need thereof, wherein the cancer is characterized by increased A_{2A}R expression or density of A_{2A}R⁺ cells.

In some embodiments, a subject has previously been identified as having increased A_{2A} receptor expression or density of A_{2A}R⁺ cells in a tumor microenvironment as compared to a reference.

In some embodiments, the present disclosure includes a method of treating cancer in a subject in need thereof, comprising administering to the subject a therapeutically effective amount of an adenosine receptor antagonist, wherein the subject has previously been identified as having increased A_{2A} receptor expression or density of A_{2A}R⁺ cells in a tumor of the subject. In some embodiments, the present disclosure includes an adenosine receptor antagonist, for use in the treatment of cancer in a subject in need thereof, wherein the cancer is characterized by increased A_{2A}R expression or density of A_{2A}R⁺ cells, and wherein the subject has previously been identified as haying increased A_{2A}R expression or density of A_{2A}R⁺ cells in a tumor of the subject. In some embodiments, a subject has previously been identified as having increased A_{2A} receptor expression or density of A_{2A}R⁺ cells in the tumor microenvironment as compared to a reference. In some embodiments, a subject has previously been identified as having increased A_{2A} receptor expression or density of A_{2A}R⁺ cells in the tumor microenvironment as compared to a reference.

In some embodiments, the present disclosure includes a method of treating cancer in a subject in need thereof, comprising:
selecting a subject with cancer having a diagnosis of an increased A_{2A} receptor expression or density of A_{2A}R⁺ cells in a tumor of the subject; and
treating the patient with an adenosine receptor antagonist.

In some embodiments, the present disclosure includes a method of selecting a subject with cancer for treatment with an adenosine receptor antagonist, comprising:
detecting the level of A_{2A}R expression or the density of A_{2A}R⁺ cells in a sample from the subject, such as in a tumor sample from the subject;
selecting the subject for treatment with an adenosine receptor antagonist based on a comparison of said level or density with a reference level or density.

In some embodiments, the present disclosure includes a method of selecting a subject with cancer for treatment with an adenosine receptor antagonist, comprising:
detecting the level of A_{2A}R expression or the density of A_{2A}R⁺ cells in a sample from the subject, such as in a tumor sample from the subject;
selecting the subject for treatment with an adenosine receptor antagonist when the level of expression or the density of A_{2A}R⁺ cells is increased.

In some embodiments, level of A_{2A}R expression or the density of A_{2A}R⁺ cells is increased as compared to a reference. In some embodiments, the level of A_{2A}R expression or the density of A_{2A}R⁺ cells is increased in the tumor microenvironment as compared to a reference.

In some embodiments, the subject is to be treated with an adenosine receptor antagonist as a first line therapy, i.e., the subject has not received prior anticancer treatment. In some embodiments, the subject is to be treated with an adenosine receptor antagonist as a second, third or more line therapy, i.e., the subject has received prior anticancer treatment with another anticancer agent.

### Combinations

In some embodiments, the present disclosure includes a method of treating cancer characterized by increased A_{2A} receptor expression or density of A_{2A}R⁺ cells in a subject in need thereof, comprising administering to the subject a combination of a therapeutically effective amount of an adenosine receptor antagonist and a therapeutically effective amount of an anticancer agent. In some embodiments, the present disclosure includes a method of treating cancer characterized by increased A_{2A} receptor expression or density of A_{2A}R⁺ cells in a tumor in a subject in need thereof comprising administering to the subject a combination of a therapeutically effective amount of an adenosine receptor antagonist and a therapeutically effective amount of an anticancer agent. In some embodiments, the present disclosure includes a combination of a therapeutically effective amount of an adenosine receptor antagonist and a therapeutically effective amount of an anticancer agent, for use in the treatment of cancer in a subject in need thereof, wherein the cancer is characterized by increased A_{2A}R expression or density of A_{2A}R⁺ cells. In some embodiments, the present disclosure includes an adenosine receptor antagonist, for use in the treatment of cancer in a subject in need thereof, wherein the cancer is characterized by increased A_{2A}R expression or density of A_{2A}R⁺ cells, and wherein the subject is further to be administered with an anticancer agent.

In one embodiment, an anticancer agent is selected from immunotherapeutic agents, chemotherapeutic agents, antiangiogenic agents, multidrug resistance-associated proteins inhibitors, radiotherapeutic agents, and any combination thereof.

In one embodiment, a combination of comprises a single anticancer agent. In another embodiment, a combination comprises a plurality of anticancer agents; preferably, two, three or four anticancer agents as defined below. In case of use of a combination of anticancer agents in a combination, an anticancer agents may be of the same class of agents or of different classes of agents. For example, a combination of an immunotherapeutic agent and of a chemotherapeutic agent may be used with a adenosine receptor antagonist.

In the context of the present disclosure, administration of an anticancer agent and an adenosine receptor antagonist may occur either simultaneously or timely staggered, either at the same site of administration or at different sites of administration, under similar or different dosage forms as further outlined below.

In the context of the present disclosure, administration of an anticancer agent and an adenosine receptor antagonist may occur either simultaneously or timely staggered, either at the same site of administration or at different sites of administration, under similar or different dosage forms as further outlined below.

In one embodiment, an anticancer agent is administered prior to, concomitant with, or subsequent to administration of an adenosine receptor antagonist. To ensure that the separate mechanisms elicited by an anticancer agent and an adenosine receptor antagonist are not negatively influenced by each other, an adenosine receptor antagonist and an anticancer agent may be administered separated in time (in a time-staggered manner), i.e. sequentially, and/or are administered at different administration sites. This means that the adenosine receptor antagonist may be administrated e.g. prior, concurrent or subsequent to an anticancer agent, or vice versa. Alternatively or additionally, an adenosine receptor antagonist and an anticancer agent may be administered at different administration sites, or at the same administration site, preferably, when administered in a time staggered manner.

In one embodiment, an adenosine receptor antagonist is to be administered prior to and/or concomitantly with an anticancer agent. In one embodiment, an adenosine receptor antagonist is to be administered prior to the day or on the same day that an anticancer agent is administered. In another embodiment, an anticancer agent is to be administered prior to and/or concomitantly with an adenosine receptor antagonist. In one embodiment, an anticancer agent is to be administered prior to the day or on the same day that an adenosine receptor antagonist is administered. In one embodiment, an adenosine receptor antagonist is to be administered prior to and/or concomitantly with an anticancer agent and continuously thereafter. In another embodiment, an anticancer agent is to be administered prior to and/or concomitantly with an adenosine receptor antagonist and continuously thereafter.

Depending on the condition to be prevented or treated and the form of administration, an anticancer agent and the adenosine receptor antagonist may be administered as a single daily dose, divided over one or more daily doses.

It will be understood that the total daily usage of adenosine receptor antagonist and anticancer agent will be decided by the attending physician within the scope of sound medical judgment. The specific dose for any particular subject will depend upon a variety of factors such as the cancer to be treated; the age, body weight, general health, sex and diet of the patient; and like factors well-known in the medical arts.

### Immunotherapeutic Agent

In one embodiment, a combination includes an immun.oth.erapeutic agent as anticancer agent.

In such case the present disclosure relates to a combination comprising:
(a) at least one adenosine receptor antagonist, and
(b) at least one immunotherapeutic agent.

In the present disclosure, “immunotherapy” refers to a therapy aiming at inducing and/or enhancing an immune response towards a specific; target, for example towards cancer cells. In such last case, it is referred to as cancer immunotherapy.

In some embodiments, immunotherapeutic agent is, for example, selected from checkpoint inhibitors, checkpoint agonists (also called T-cell agonists), IDO inhibitors, PI3K inhibitors, adenosine receptor inhibitors, adenosine-producing enzymes inhibitors, CD40 agonists, variants, immune cells (for conducting adoptive transfer), therapeutic vaccines, and combinations thereof. In a specific embodiment, the immunotherapeutic agent is a checkpoint inhibitor.

In one embodiment, an immunothera.peutic agent to be combined with adenosine receptor antagonist as described hereinabove comprises or consists of checkpoint inhibitors, checkpoint agonists, IDO inhibitors, PI3K inhibitors, adenosine receptor inhibitors, adenosine-producing enzymes inhibitors, CD40 agonists, IL2 variants, immune cells (for conducting adoptive transfer), therapeutic vaccines, or any mixes thereof.

### Checkpoint Inhibitors

In one embodiment, a combination includes at least one checkpoint inhibitor as immunotherapeutic agent.

In some embodiments, checkpoint inhibitors (CP1), that may also be referred to as immune checkpoint inhibitors (IQ, block the interactions between inhibitory receptors expressed on T cells and their ligands. As a cancer treatment, use of checkpoint inhibitor aims at preventing the activation of inhibitory receptors expressed on T cells by ligands expressed by a tumor. Use of checkpoint inhibitors thus aims at preventing, inhibition of T cells present in the tumor, i.e., tumor infiltrating T cells, and thus at enhancing the subject immune response towards a tumor.

Thus, a combination of the present disclosure can restore immune functions in tumor environments by using as a first component an A_{2A}R inhibitor, and to antagonize checkpoint pathway signaling by preferably inhibiting or suppressing signal transduction by using as second component a checkpoint inhibitor as immunotherapeutic agent.

Examples of checkpoint inhibitors include, without being limited to:
inhibitors of the cell surface receptor PD-1 (programmed cell death protein 1), also known as CD279 (cluster differentiation 279);
inhibitors of the ligand PD-L1 (programmed death-ligand 1), also known as CD274 (cluster of differentiation 274) or B7-H1 (B7 homolog 1);
inhibitors of the cell surface receptor CTLA4 or CTLA-4 (cytotoxic T-lymphocyte-associated protein 4), also known as CD152 (cluster of differentiation 152);
inhibitors of LAG-3 (lymphocyte-activation gene 3), also known as CD223 (cluster differentiation 223);
inhibitors of TIM-3 (T-cell immunoglobulin and mucin-domain containing-3), also known as HAVCR2 (hepatitis A virus cellular receptor 2) or CD366 (cluster differentiation 366);
inhibitors of TIGIT (T cell immunoreceptor with Ig and ITIM domains), also known as VSIG9 (V-Set And Immunoglobulin Domain-Containing Protein 9) or VSTM3 (V-Set And Transmembrane Domain-Containing Protein 3);
inhibitors of BTLA (B and T lymphocyte attenuator), also known as CD272 (cluster differentiation 272);
inhibitors of CEACAM-1 (carcinoembryonic antigen-related cell adhesion molecule 1) also known as CD66a (cluster differentiation 66a); and
inhibitors of GITR (glucocorticoid-induced TNFR-related protein) also known as TNFRSF18 (tumor necrosis factor receptor superfamily member 18) or AITR (activation-inducible TNFR family receptor).

In one embodiment, a checkpoint inhibitor is selected from the group comprising or consisting of inhibitors of PD-1, inhibitors of PD-L1, inhibitors of CTLA4, inhibitors of LAG-3, inhibitors of TIM-3, inhibitors of TIGIT, inhibitors of BTLA, inhibitors of CEACAM-1, inhibitors of GITR and any mixtures thereof.

In one embodiment, a checkpoint inhibitor is selected from the group comprising or consisting of inhibitors of PD-1, inhibitors of PD-L1, inhibitors of CTLA-4, inhibitors of TIGIT and any mixtures thereof.

In one embodiment, a checkpoint inhibitor is selected from the group comprising or consisting of inhibitors of PD-1, inhibitors of PD-L1, inhibitors of CTLA-4 and any mixtures thereof.

In one embodiment, a checkpoint inhibitor is an inhibitor of PD-1, also referred to as an anti-PD-1. inhibitors of PD-1 may include antibodies targeting PD-1, in particular monoclonal antibodies, and non-antibody inhibitors such as small molecule inhibitors.

Examples of inhibitors of PD-1 include, without being limited to, pembrolizumab, nivolurnab, cemiplimab, tislelizumab, spartalizumab, ABBV-181, JNJ-63723283, BI 754091, MAG012, TSR-042, AGEN2034. Pembrolizumab is also known as MK-3475, MK03475, lambrolizumab, or SCH-900475. The trade name of pembrolizumab is Keytruda®. Nivolumab is also known as ONO-4538, BMS-936558, MDX1106, or GTPL7335. The trade name of nivolumab is Opdivo®. Cemiplimab is also known as REGN2810 or REGN-2810. Tislelizumab is also known as BGB-A317. Spartalizumab is also known as PDR001 or PDR-001.

In one embodiment, a checkpoint inhibitor is selected from the group comprising or consisting of pembrolizumab, nivolumab, cemiplimab, tislelizumab, spartalizumab. ABBV-181, JNJ-63723283, BI 754091, MAG012, TSR-042, AGEN2034, and any mixtures thereof.

In one embodiment, a checkpoint inhibitor is an inhibitor of PD-L1, also referred to as an anti-PD-L1 Inhibitors of PD-L1 may include antibodies targeting PD-L1, in particular monoclonal antibodies, and non-antibody inhibitors such as small molecule inhibitors.

Examples of inhibitors of PD-L1 include, without being limited to, avelumab, atezolizumab, durvalumab and LY3300054. Avelumab is also known as MSB0010718C, MSB-0010718C, MSB0010682, or MSB-0010682. The trade name of avelumab is Bavenciot®. Atezolizumab is also known as MPDL3280A (clone YW243.55.S70), MPDL-3280A, RG-7446 or RG7446, The trade name of atezolizumab is Tecentriq®. Durvalumab is also known as MEDI4736 or MEDI-4736. The trade name of durvalumab is Imfinzi®.

In one embodiment, a checkpoint inhibitor is selected from the group comprising or consisting of avelumab, atezolizumab, durvalumab, LY3300054, and any mixtures thereof.

In one embodiment, a checkpoint inhibitor is an inhibitor of CTLA-4, also referred to as an anti-CTLA-4.

Inhibitors CTLA-4 may include antibodies targeting CTLA-4, in particular monoclonal antibodies, and non-antibody inhibitors such as small molecule inhibitors.

Examples of inhibitors of CTLA-4 include, without being limited to, ipilimumab and tremelimumab. Ipilimumab is also known as BMS-734016, MDX-010, or MDX-101. The trade name of ipilimumab is Yervoy®. Tremelimumab is also known as ticilimumab, CP-675, or CP-675,206.

In one embodiment, at least one checkpoint inhibitor is selected from the group comprising or consisting of ipilimumab, tremelimumab, and any mixtures thereof.

In one embodiment, a checkpoint inhibitor is an inhibitor of TIGIT, also referred to as an anti-TIGIT.

In one embodiment of various methods, pharmaceutical compositions, kits, or uses provided herein, an anti-human TIGIT monoclonal antibody or antigen binding fragment thereof is BMS-986207 (Bristol-Myers Squibb, New York, NY).

In another embodiment of various methods, pharmaceutical compositions, kits, or uses provided herein, an anti-human TIGIT monoclonal antibody or antigen binding fragment thereof is OMP-313M32 (OncoMed Pharmaceuticals, Redwood city, CA).

In another embodiment of various methods, pharmaceutical compositions, kits, or uses provided herein, an anti-human TIGIT monoclonal antibody or antigen binding fragment thereof MK-7684 (Merck & Co., Kenilworth, NJ).

In yet another embodiment of various methods, pharmaceutical compositions, kits, or uses provided herein, an anti-human TIGIT monoclonal antibody or antigen binding fragment thereof is MTIG7192A (also known as RG6058, U.S. Publ. No. 2017/0088613).

In still another embodiment of various methods, pharmaceutical compositions, kits, or uses provided herein, an anti-human TIGIT monoclonal antibody or antigen binding fragment thereof is PTZ-201 (Potenza Therapeutics, Cambridge, MA; also known as ASP8374, Astellas Pharma, Tokyo, Japan).

In another embodiment of various methods, pharmaceutical compositions, kits, or uses provided herein, an anti-human TIGIT monoclonal antibody or antigen binding fragment thereof COM902 (Compugen LTD, Holm, IL).

In yet another embodiment of various methods, pharmaceutical compositions, kits, or uses provided herein, an anti-human TIGIT monoclonal antibody or antigen binding fragment thereof is described in WO2018/160704 (Seattle Genetics, Seattle, WA).

In yet another embodiment of various methods, pharmaceutical compositions, kits, or uses provided herein, an anti-human TIGIT monoclonal antibody or antigen binding fragment thereof is described in WO2019/023504 (Iteos Therapeutics). In certain preferred embodiments, an anti-human TIGIT antibody or antigen binding fragment comprises a combination of HCDR1, HCDR2, HCDR3, LCDR1, LCDR2 and LCDR3, wherein:

HCDR1 comprises or consists of SEQ ID NO: 2,
(YTFTSYYMH)
HCDR2 comprises or consists of SEQ ID NO: 3,
(VIGPSGASTSYAQKFQG)
HCDR3 comprises or consists of SEQ ID NO: 4,
(ARDHSDYWSGIMEV)
LCDR1 comprises or consists of SEQ ID NO: 5,
(RASQSVRSSYLA)
LCDR2 comprises or consists of SEQ ID NO: 6,
(GASSRAT)
and
LCDR3 comprises or consists of SEQ ID NO: 7.
(QQYFSPPWT)

### Checkpoint Agonists (T-Cell Agonists)

In one embodiment, a combination of the present disclosure includes at least one checkpoint agonist (also referred to as T-cell agonist) as immunotherapeutic agent.

T-cell agonists act by activating stimulatory receptors expressed on immune cells, such as cells. As used herein, the term “stimulatory receptors” refer to receptors that induce a stimulatory signal upon activation, and thus lead to an enhancement of the immune response. As a cancer treatment, T-cell agonist therapy aims at activating stimulatory receptors expressed on immune cells present in a tumor. In particular, T-cell agonist therapy aims at enhancing the activation of T cells present in a tumor, i.e., tumor infiltrating T cells, and thus at enhancing the subject immune response towards a tumor.

Examples of T-cell agonists include, without being limited to:
agonists of CD137 (cluster differentiation 137) also known as 4-1BB or TNFRS9 (tumor necrosis factor receptor superfamily, member 9);
agonists of OX40 receptor also known as CD134 (cluster differentiation 134) or TNFRSF4 (tumor necrosis factor receptor superfamily, member 4).

In one embodiment, a checkpoint agonist is selected from the group comprising or consisting of agonists of CD137, agonists of OX40 and any mixtures thereof.

Examples of agonists of CD137 include, without being limited, utomilumab and urelumab.

### IDO Inhibitors

In one embodiment, a combination of the present disclosure includes at least one inhibitor of indoleamine-2,3-dioxygenase (IDO) as immunotherapeutic agent.

Indolearnine 2,3-dioxygenase enzyme catalyzes the first and rate-limiting step of L-tryptophan (Trp) catabolism. IDO is implicated in immune modulation through its ability to limit T well function and engage mechanisms of immune tolerance, IDO activity in a tumor serves to impair anti-tumor responses. Inhibiting IDO thus enables to restore tumor immune surveillance.

Examples of IDO inhibitors include beta-carboline (also known as norharmane), rosmarinic acid, 1-methyl-L-tryptophan (also known as L-1-MT), epacadostat, navoximod or those disclosed in WO2015/173764, and more preferably those a formula II, II′ or II″.

In a preferred embodiment, an IDO inhibitor is selected among those disclosed in WO2015/173764, and more preferably those of formula II, II′or II″.

### PI3K Gamma Inhibitors

In one embodiment, a combination of the present disclosure includes at least one PI3K inhibitor as immunotherapeutic agent.

A phosphoinositide 3-kinase inhibitor (PI3K inhibitor) is a class of medical drug that functions by inhibiting one or more of the phosphoinositide 3-kinase enzymes, which are part of the PI3K/AKT/mTOR pathway, an important signaling pathway for many cellular functions such as growth control, metabolism and translation initiation. Many types of cancers have activated PI3K pathway, which prohibit tumor cel is from cell death.

There are a number of different classes and isoforms of PI3Ks. Class 1 PI3Ks have a catalytic subunit known as p110, with four types (isoforms)—p110 alpha, p110 beta, p110 gamma and p110 delta.

In a preferred embodiment, a PI3K inhibitor is a PI3K-gamma inhibitor.

Examples of PI3K inhibitors include wortmannin, LY294002, demethoxyviridon, hibiscone C, Idelalisib, Copanlisib, Duvelisib, Taselisib, Buparlisib, Alpelisib, Umbralisib, Dactolisib, Voxtalisib, IPI-549, RP6530, IC87114 and TG100-115.

Examples of PI3K-gamma inhibitors include Copanlisib, Duvelisib, IPI-549, RP6530, IC87114 and TG100-115.

### CD40 Agonists

In one embodiment, a combination of the present disclosure includes at least one CD40 agonist as immunotherapeutic agent.

CD40 is a cell surface receptor member of the tumor necrosis factor (TNF) receptor superfamily. It mediates both indirect tumor cell killing through the activation of the immune system and direct tumor cell apoptosis. Similar to the endogenous CD40 ligand (CD40L or CD154), CD40 agonists bind to CD40 on a variety of immune cell types. This triggers the cellular proliferation and activation of antigen-presenting cells (APCs), and activates B-cells, and effector and memory T cells. This results in an enhanced immune response against tumor cells.

Examples of CD40 agonists include CD40 agonistic antibodies and recombinant CD40 agonists (ie proteins, but not antibodies). Examples of CD40 agonistic antibodies include selicrelumab (formely known as RO7009789 and CP-870,8931, APX005M, JNJ-64457107 (formerly ADC-1013), SEA-CD40, ChiLob 7/4. CDX-1140H, dacetuzumab (SGN-40) and ABBV-428. Examples of recombinant CD40 agonists include MEDI5083 and HERA-CD40L.

### Adenosine-Producing Enzymes Inhibitors

In one embodiment, a combination of the present disclosure includes at least one adenosine-producing enzymes inhibitor as immunotherapeutic agent.

Ectonucleotidases are families of nucleotide metabolizing enzymes that metabolize nucleotides to nuclcosides. Subfamilies of ectonucleotidases include: CD39/NTPDases (ecto-nucleotide triphosphate diphosphohydrolases), nucleotide pyrophosphatase/phosphodiesterase (NPP)-type ecto-phosphodiesterases, alkaline phosphatases and ecto-5′-nucleotidases/CD73.

Among other functions, ectonucleotidases generate extracellular adenosine, a first step involving the conversion of ATP/ADP to AMP, carried out by ENTPD1 also known as CD39 a second step involves the conversion of AMP to adenosine. It is carried out by NT5E, also known as CD73. Thus ectonucleotidases are adenosine-producing enzymes.

Examples of adenosine-producing enzymes inhibitors include:
inhibitors of CD39, also known as ENTPD1 or Ecto-nucleoside triphosphate diphosphohydrolases (EC 3.6.1.5, apyrase),
inhibitors of CD73, also known as 5′-nucleotidase (5′-NT) or ecto-5′-nucleotidase or NT5E,
inhibitors of Ecto-nucleotide pyrophosphatase/PDEs (EC 3.6.1.9 and EC 3.1.4.1) and
inhibitors of alkaline phosphatases (APs; EC 3,1.3.1),
inhibitors of C1138, also known as cyclic ADP ribose hydrolase or ADP-ribosyl cyclase/cyclic ADP-ribose (cADPR) hydrolase).

Examples of adenosine-producing enzymes inhibitors include IPH-5201, A001485, SRF617, ARL67156, POM-1, IPH15301, A000830, A001190, A001421, SRF373/NZV930, Darutumumab. More precisely, examples of CD39 inhibitors include IPE15201, .A001485, SRF617, ARL67156 and POM-1, examples of CD73 inhibitors include IPH5301, A000830, A001190, A001421 and SRF373/NZV930; and examples of CD38 inhibitors include Darutumumab.

### IL2 Variants

In one embodiment, a combination of the present disclosure includes at least one IL2 variant as immunotherapeutic agent.

Interleulin-2 (IL-2) is a powerful immune growth factor that plays an important role in sustaining T cell response. The potential of IL-2 in expanding T cells without loss of functionality has led to its early use in cancer immunotherapy.

Examples of IL2 variants include recombinant, PEGylated and/or mutated IL2 variants, such as for example aldesleukin, monomethoxy PEG IL2, NKTR-214, 1MDNA-109, RO6874281 and ALKS-4230.

### Immune Cells—Adoptive Cell Transfer

According to one embodiment, an immunotherapeutic agent is immune cells to be used in an adoptive transfer of cells, also referred to as adoptive cell therapy (both also referred to as ACT), particularly an adoptive transfer of T cells, also referred to as adoptive T cell therapy.

As used herein, an adoptive transfer of cells or adoptive cell therapy is defined as the transfer, for example as an infusion, of immune cells to a subject. As a cancer treatment, an adoptive transfer of immune cells to a subject aims at enhancing the subject immune response towards the cancer cells.

In one embodiment, an immune cells are T cells, in particular effector T cells. Examples of effector T cells include CD4+ T cells and CD8+ T cells.

In one embodiment, a transferred T cells are cytotoxic cells. Examples of cytotoxic T cells include CD8+ T cells and natural killer (NK) cells, in particular natural killer (NK) T cells.

In one embodiment, a transferred immune cells as described hereinabove are antigen-specific cells. In one embodiment, a transferred immune cells as described hereinabove are antigen-specific immune cells, wherein said antigen is specifically and/or abundantly expressed by cancer cells. In one embodiment, a transferred immune cells as described hereinabove are cancer-specific immune cells, in other words the transferred immune cells as described hereinabove specifically recognize cancer cells through an antigen specifically and/or abundantly expressed by said cancer cells. In one embodiment, a transferred immune cells as described hereinabove are cancer-specific effector T cells. In one embodiment, a transferred immune cells as described hereinabove are cancer-specific CD8+ effector T cells, in particular cancer-specific cytotoxic CD8+ cells. In one embodiment, a transferred immune cells as described hereinabove are cancer-specific, cytotoxic cells. In one embodiment, a transferred immune cells as described hereinabove are cancer-specific NK cells. In one embodiment, a transferred immune cells as described hereinabove are tumor-specific immune cells, in other words a transferred immune cells as described hereinabove specifically recognize tumor cells through an antigen specifically and/or abundantly expressed by said tumor cells. In one embodiment, a transferred immune cells as described hereinabove are tumor-specific effector T cells. In one embodiment, a transferred immune cells as described hereinabove are tumor-specific CD8+ effector T cells, in particular tumor-specific cytotoxic cells. In one embodiment, a transferred immune cells as described hereinabove are tumor-specific cytotoxic cells. In one embodiment, a transferred immune cells as described hereinabove are tumor-specific NK cells.

In one embodiment, a transferred immune cells as described hereinabove are autologous immune cells, in particular autologous cells. In another embodiment, a transferred immune cells as described hereinabove are allogenic (or allogenous) immune cells, in particular allogenic NK cells.

Methods to isolate T cells from a subject, in particular antigen-specific T cells, e.g., tumor-specific T cells, are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71). Methods to expand cells ex vivo are well-known in the art (see for example Rosenberg & Restifo, 2015, Science 348, 62-68; Prickett et al., 2016, Cancer immunol Res 4, 669-678 or Hinrichs & Rosenberg, 2014, Immunol. Rev 257, 56-71). Protocols for infusion of T cells in a subject, including pre-infusion conditioning regimens, are well-known in the art (see for example Rosenberg & Restifo, 2015, Science 348, 62-68; Prickett et al., 2016, Cancer immunol Res 4, 669-678; or Hinrichs & Rosenberg, 2014, Immunol Rev 257, 56-71).

In one embodiment, immune cells are CAR immune cells, in particular a CAR T cells, in the context respectively of CAR immune cell therapy and CAR T cell therapy.

As used herein, CAR immune cell therapy is an adoptive cell therapy wherein transferred cells are immune cells as described hereinabove, such as cells or NK cells, genetically engineered to express a chimeric antigen receptor (CAR). As a cancer treatment, the adoptive transfer of CAR immune cells to a subject aims at enhancing the subject immune response towards the cancer cells.

CARs are synthetic receptors consisting of a targeting moiety that is associated with one or more signaling domains in a single fusion molecule or in several molecules. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are usually derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Thus, signaling domains from co-stimulatory molecules including CD28, OX-40 (CD134), and 4-1BB (CD137) have been added alone (second generation) or in combination (third generation) to enhance survival and increase proliferation of CAR modified T cells.

Thus, in one embodiment, transferred T cells as described hereinabove are CAR T cells. Expression of a CAR allows the T cells to be redirected against a selected antigen, such as an antigen expressed at the surface of cancer cells. In one embodiment, transferred CAR T cells recognize a tumor-specific antigen.

In another embodiment, transferred NK cells as described hereinabove are CAR NK cells. Expression of a CAR allows the NK cells to be redirected against a selected antigen, such as an antigen expressed at the surface of cancer cells. In one embodiment, the transferred CAR NK cells recognize a tumor-specific antigen.

In one embodiment, CAR immune cells as described hereinabove are autologous CAR immune cells, in particular autologous CAR T cells. In another embodiment, CAR immune cells as described hereinabove are allogenic (or allogenous) CAR immune cells, in particular allogenic CAR NK cells.

### Therapeutic Vaccines

According to one embodiment, an immunotherapeutic agent is a therapeutic vaccine (sometimes also referred to as a treatment vaccine).

As used herein, a therapeutic vaccine is defined as the administration of at least one tumor-specific antigen (e.g., synthetic long peptides or SLP), or of the nucleic acid encoding said tumor-specific antigen; administration of recombinant viral vectors selectively entering and/or replicating in tumor cells; the administration of tumor cells; and/or administration of immune cells dendritic cells) engineered to present tumor-specific antigens and trigger an immune response against these antigens.

As a cancer treatment, therapeutic vaccines aim at enhancing a subject immune response towards the tumor cells.

Examples of therapeutic vaccines aiming at enhancing a subject immune response towards the tumor cells include, without being limited to, viral-vector based therapeutic vaccines such as adenoviruses (e.g., oncolytic adenoviruses), vaccinia viruses (e.g., modified vaccinia Ankara (MVA)), alpha viruses (e.g., Semliki Forrest Virus (SFV)), measles virus, Herpes simplex virus (HSV), and coxsackievirus; synthetic long peptide (SLP) vaccines; and dendritic cell vaccines.

### Chemotherapeutic Agent

In one embodiment, a combination of the present disclosure includes at least one chemotherapeutic agent as anticancer agent.

A chemotherapeutic agent is for example selected from anticancer alkylating agents, anticancer antimetabolites, anticancer antibiotics, plant-derived anticancer agents, anticancer platinum coordination compounds, Parp inhibitors, and-hormone-sensitive cancer agents and any combination thereof.

In one embodiment, a chemotherapeutic agent to be combined with the A_{2A}R inhibitor of Formula (I) as described hereinabove comprises or consists of anticancer alkylating agents, anticancer antimetabolites, anticancer antibiotics, plant-derived anticancer agents, anticancer platinum coordination compounds, Parp inhibitors, and-hormone-sensitive cancer agents and any combination thereof.

### Anticancer Alkylating Agent

In one embodiment, a combination of the present disclosure includes at least one anticancer alkylating agent as chemotherapeutic agent.

An anticancer alkylating agent refers to an alkylating agent having anticancer activity, and the term “alkylating agent” herein generally refers to an agent giving an alkyl group in the alkylation reaction in which a hydrogen atom of an organic compound is substituted with an alkyl group.

Examples of anticancer alkylating agents include nitrogen mustard N-oxide, cyclophosphamide, ifosfamide, melphalan, busulfan, mitobronitol, carboquone, thiotepa, rammustine, nimustine, temozolomide and carmustine.

### Anticancer Antimetabolite

In one embodiment, a combination of the present disclosure includes at least one anticancer antimetabolite as chemotherapeutic agent.

An anticancer antimetabolite refers to an antimetabolite having anticancer activity, and the term “antimetabolite” herein includes, in a broad sense, substances which disturb normal metabolism and substances which inhibit the electron transfer system to prevent the production of energy-rich intermediates, due to their structural or functional similarities to metabolites that are important for living organisms (such as vitamins, coenzymes, amino acids and saccharides).

Examples of anticancer antimetabolites include methotrexate, 6-mercaptopurine riboside, mercaptopurine, 5-fluorouracil (also called “5-FU”), tegafur, doxiflurdine, carrnofur, cytarabine cytarabine ocfosfate, enocitahine, S-1, gemcitabine, fludarabine and pemetrexed disodium. Preferably the anticancer antimetabolite is selected from 5-FU, gemcitabine and pemetrexed.

### Anti Cancer Antibiotic

In one embodiment, a combination of the present disclosure includes at least one anticancer antibiotic as chemotherapeutic agent.

An “anticancer antibiotic” refers to an antibiotic having anticancer activity, and the “antibiotic” herein includes substances that are produced by microorganisms or by partial or total synthesis, and derivatives thereof, and inhibit cell growth and other functions of microorganisms and of other living organisms.

Examples of anticancer antibiotic include actinomycin D, doxorubicin, daunorubicin, neocarzinostatin, bleomycin, peplomycin, mitomycin C, aclarubicin, pirarubicin, epirubicin, zinostatin stimalamer, idarubicin, sirolimus and valrabicin. Preferably, thenanticancer antibiotic is doxorubicin.

### Plant-Derived Anticancer Agent

In one embodiment, a combination of the present disclosure includes at least one plant-derived anticancer agent as chemotherapeutic agent.

A “plant-derived anticancer agent” as used in the specification includes compounds having anticancer activities which originate from plants, or compounds prepared by applying chemical modification to the foregoing compounds.

Examples of plant-derived anticancer agent include vincristine, vinblastine, vindesine, etoposide, sobuzoxane, docetaxel, paclitaxel, nab-paclitaxel and vinorelbine. Preferably, the plant-derived anticancer agent is docetaxel.

### Anticancer Platinum Coordination Compound

In one embodiment, a combination of the present disclosure includes at least one anticancer platinum coordination compound as chemotherapeutic agent.

An “anticancer platinum coordination compound” refers to a platinum coordination compound having anticancer activity, and the term “platinum coordination compound” herein refers to a platinum coordination compound which provides platinum in ion form.

Preferred platinum compounds include cisplatin; cis-diamminediaquoplatinum (O)-ion; chloro(diethylenetriamine)-platinum (II) chloride; dichloro(ethylenediamine)-platinum (II); diamine(1,1-cyclobutanedicarboxylato) platinum (II) (carboplatin); spiroplatin iproplatin; diamine(2-ethylmalonato)platinum (II); ethylenediaminemalonatoplatinum (H); aqua(1,2-diaminodicyclohexane)sulfatoplatinum (II); aqua(1,2-diaminodicyclohexane)malonatoplatinum (II); (1,2-diaminocyclohexane)malonatoplatinum (II); (4-carboxyplithalato)(1,2-diaminocyclohexane)platinum (II); (1,2-diaminocyclohexane)-(isocitrato)platinum (II); (1,2-diaminocyclobexane)oxalatoplatinum (II); ormaplatin; tetraplatin; carboplatin, nedaplatin and oxaliplatin. Preferably the anticancer platinum coordination compound is selected from carboplatin and oxaliplatin.

### Parp Inhibitors

In one embodiment, a combination of the present disclosure includes at least one Parp inhibitor as chemotherapeutic agent.

A “Parp inhibitor” refers to an inhibitor of the enzyme poly ADP ribose polymerase (PARP). This enzyme is important for repairing single-strand breaks in the DNA. If such breaks remain unrepaired until DNA is replicated, then the replication can cause double strand breaks to form. PARP inhibitors thus enable to cause multiple double strand breaks to form in tumors, leading to the death of the tumor cells.

Examples of Parp inhibitors include olaparib, rucaparib, niraparib, veliparib, pamiparib, iniparib, and talazoparib.

### Anti-Hormone-Sensitive Cancer Agent

In one embodiment, a combination of the present disclosure includes at least one anti-hormone-sensitive cancer agent as chemotherapeutic agent.

An “anti-hormone-sensitive cancer agent” refers to an anticancer agent having an activity against hormone-sensitive cancers. Examples of anti-hormone-sensitive cancer agents include anti-androgens, GnRH agonists and GnRH antagonists.

“Anti-androgens” refer to a class of drugs that prevent androgens like testosterone and dihydrotestosterone (DHT) from mediating their biological effects in the body. Anti-androgens may be used for example to treat prostate cancer. Examples of anti-androgens include bicalutamide, flutamide, nilutamide, apalutamide, enzalutamide and abiraterone.

“Gonadotropin-releasing hormone agonists” (GnRH agonist) refer to a class of drugs which affects gonadotropins and sex hormones. They may be used to lower sex hormone levels in the treatment of hormone-sensitive cancers such as prostate cancer and breast cancer. Examples of GnRH agonists include goserelin, leuprorelin and triptorelin.

“Gona.dotropin-releasing hormone antagonists” (GnRH antagonist) refer to a class of drugs that antagonize the action of gonadotropin-releasing hormone (GnRH). They may be used for example in the treatment of prostate cancer. An example of GnRH antagonist is degarelix.

### Combinations of Chemotherapeutic Agents

Combinations of chemotherapeutic agents may be used as a second component of a combination of the present disclosure.

For example, a combination known as folfox may be used. Folfox comprises the combined use of fluorouracil (antimetabolite), oxaliplatin (platinum compound) and folinic acid (chemoprotectant).

A combination consisting of carboplatin (platinum compound) and paclitaxel (plant-derived agent) may alternatively be used. Another example is a combination consisting of gemcitabine (antimetabolite) and nab-paclitaxel (plant-derived agent).

In one embodiment, a combination of chemotherapeutic agents is selected from:
(i) a combination consisting of folinic acid, fluorouracil and oxaliplatin (folfox);
(ii) a combination consisting of carboplatin and paclitaxel; and
(iii) a combination consisting of gemcitabine and nab-paclitaxel.

### Antiangiogenic Agent

In one embodiment, a combination of the present disclosure includes at least one anti angiogenic agent as anticancer agent.

Angiogenesis, i.e. growth of new blood vessels, plays an important role in the development of tumors and the progression of malignancies. Inhibiting angiogenesis has been shown to suppress tumor growth and metastasis. The most prominent target of antiangiogenic agents is vascular endothelial growth factor (VEGF) and its receptors. Several other factors are of interest as well, including integrins, matrix metalloproteinases, and endogenous antiangiogenic factors.

Antiangiogenic agents thus include VEGF inhibitors, integrins inhibitors and matrix metal loproteinases inhibitors.

Examples of antiangiogenic agents include Ramucirumab, IMC-18F1; Bevacizumab, Zivaflibercept, Sorafenib, Sunitinib, Axitinib, Nintedanib, Regorafenib, Pazobanib, Cab ozantinib, Vandetanib and Thalidomide. In a specific embodiment, the antiangiogenic agent is a VEGF inhibitor, for example Ramucirumab.

### Multidrug Resistance-Associated Proteins Inhibitors

In one embodiment, a combination of the present disclosure includes at least one multidrug resistance-associated protein inhibitor as anticancer agent.

Multidrug resistance-associated proteins (MRP/ABCC) are a subfamily of ATP-binding cassette transporters, which are capable of actively pumping a wide variety of organic anionic compounds across the plasma membrane against their concentration gradient. These proteins are involved in multi-drug resistance by transporting a wide variety of drugs outside cells, among which anticancer drugs. Inhibiting multidrug resistance-associated proteins can thus improve efficacy of anticancer drugs.

Examples of multidrug resistance-associated protein inhibitor include inhibitors of MRP4/ABCC4, inhibitors of MRP5/ABCC5 and inhibitors of MRP8/ABCC11.

### Radiotherapeutic Agents—Radiation Therapy

In one embodiment, a combination of the present disclosure includes at least one radiotherapeutic agent as anticancer agent.

“Radiation therapy” refers to a method of treatment of cancer employing various radiations such as X-ray, γ-ray, neutron ray, electron beam, proton beam and radiation sources. It is used as part of cancer treatment to control or kill malignant cells. Radiation therapy may be curative in a number of types of cancer if they are localized to one area of the body. It may also be used as part of adjuvant therapy, to prevent tumor recurrence after surgery to remove a primary malignant tumor.

The three main divisions of radiation therapy are: external beam radiation therapy (EBRT or XRT); brachytherapy or sealed source radiation therapy; and systemic radioisotope therapy (RIT) or unsealed source radiotherapy. The differences relate to the position of the radiation source; external is outside the body, brachytherapy uses sealed radioactive sources placed precisely in the area under treatment, and systemic radioisotopes are given by infusion or oral ingestion. Particle therapy is a special case of external beam radiation therapy where the particles are protons or heavier ions. Radiations may be delivered by a linear accelerator.

Systemic radioisotope therapy (RIT) is a form of targeted therapy. Targeting can be due to the chemical properties of the isotope such as radioiodine which is specifically absorbed by the thyroid gland a thousand fold better than other bodily organs. Targeting can also be achieved by attaching the radioisotope to another molecule or antibody to guide it to the target tissue, forming a radiopharmaceutical agent.

In order to enhance the radiosensitivity of the cancer, radiosensitizing agents may be administered during a radiation therapy. Examples of radiosensitizing agents include: Cisplatin, Nimorazole, and Cetuximab.

Thus, in one embodiment, radiotherapeutic agent is selected from sealed radiation sources, radioisotopes, radiopharmaceutical agents, radiosensitizing agents and the like useful in the course of radiation therapy.

In another embodiment, the present disclosure also provides the use of adenosine receptor antagonist as described above, in combination with radiation therapy, including radiation therapy performed by external beam radiations or X-ray radiations; brachytherapy; and systemic radioisotope therapy.

### Specific Combinations

In one embodiment, a combination of the present disclosure comprises at least one A_{2A}R inhibitor as defined above and at least one anticancer agent as defined above.

In a specific embodiment, a combination of the present disclosure comprises at least one A_{2A}R inhibitor as defined above and at least one immunotherapeuti c agent as defined above.

In a specific embodiment, a combination of the present disclosure comprises at least one A_{2A}R inhibitor as defined above and at least one checkpoint inhibitor as defined above, preferably an inhibitor of PD-1, PD-L1, CTLA-4 or of TIGIT, or any mixture thereof.

In a specific embodiment, a combination of the present disclosure comprises at least one A_{2A}R inhibitor as defined above and at least one adenosine-producing enzymes inhibitor as defined above, preferably at least one inhibitor of CD39, such as for example ARL67156 and POM-1.

In a specific embodiment, a combination of the present disclosure comprises at least one A_{2A}R inhibitor as defined above and at least one chemotherapeutic agent as defined above.

In a specific embodiment, a combination of the present disclosure comprises at least one A_{2A}R inhibitor as defined above and at least one anticancer antibiotic as defined above, such as for example doxorubicin.

In a specific embodiment, a combination of the present disclosure comprises at least one A_{2A}R inhibitor as defined above and at least one anticancer platinum coordination compound as defined above, such as for example oxaliplatin.

t a specific embodiment, a combination of the present disclosure comprises at least one A_{2A}R inhibitor as defined above, at least one immunotherapeutic agent as defined above and at least one chemotherapeutic agent as defined above.

In a specific embodiment, a combination of the present disclosure comprises at least one A_{2A}R inhibitor as defined above, at least one checkpoint inhibitor as defined above and at least one chemotherapeutic agent as defined above. In a specific embodiment, the combination of the present disclosure comprises at least one A_{2A}R inhibitor as defined above, at least one inhibitor of CTLA-4 or TIGIT and at least one chemotherapeutic agent as defined above. In a specific embodiment, the combination of the present disclosure comprises at least one A_{2A}R inhibitor as defined above, at least one checkpoint inhibitor as defined above and at least one. In a specific embodiment, the combination of the present disclosure comprises at least one A_{2A}R inhibitor as defined above, at least one inhibitor of PD-L1, CITA-4 or TIGIT as defined above and at least one anticancer antibiotic as defined above, such as for example doxorubicin.

In a specific embodiment, a combination of the present disclosure comprises at least one A_{2A}R inhibitor as defined above and at least two checkpoint inhibitor as defined above. In a specific embodiment, the combination of the present disclosure comprises at least one A_{2A}R inhibitor as defined above, at least one inhibitor of PD-L1 as defined above and at least one inhibitor of TIGIT as defined above.

### Diseases and Disorders

In some embodiments, the present disclosure includes the methods of treating proliferative disorders, including cancers. In some embodiments, the present disclosure includes a compound for use in the treatment and/or prevention of proliferative disorders, including cancers. Thus, in one embodiment, the present disclosure provides use of a compound for the manufacture of a medicament for treating and/or preventing cancer. The present disclosure also provides a method of treatment of cancer, which comprises administering to a mammal species in need thereof a therapeutically effective amount of a compound.

The present disclosure also provides for a method for delaying in patient the onset of cancer comprising the administration of a pharmaceutically effective amount of a compound of the disclosure to a patient in need thereof.

Various cancers are known in the art. Cancers that can be treated using methods of the disclosure include solid cancers and non-solid cancers, especially benign and malignant solid tumors and benign and malignant non-solid tumors. Cancer may be metastatic or non-metastatic. The cancer may be may be familial or sporadic.

In some embodiments, cancer is a solid cancer. As used herein, the term “solid cancer” encompasses any cancer (also referred to as malignancy) that forms a discrete tumor mass, as opposed to cancers (or malignancies) that diffusely infiltrate a tissue without forming a mass.

Examples of solid tumors include, but are not limited to: biliary tract cancer, brain cancer (including glioblastomas and medulloblastomas), breast cancer, carcinoid, cervical cancer, choriocarcinoma, colon cancer, colorectal cancer, endometrial cancer, esophageal cancer, gastric cancer, glioma, head and neck cancer, intraepithelial neoplasms (including Bowen's disease and Paget's disease), liver cancer, lung cancer, neuroblastomas, oral cancer (including squamous cell carcinoma), ovarian cancer (including those arising from epithelial cells, stromal cells, germ cells and mesenchymal cells), pancreatic cancer, prostate cancer, rectal cancer, renal cancer (including adenocarcinoma and Wilms tumor), sarcomas (including leiomyosarcoma, rhabdomyosarcoma, liposarcoma, fibrosarcoma and osteosarcoma), skin cancer (including melanoma, Kaposi's sarcoma, basocellular cancer and squamous cell cancer), testicular cancer including germinal tumors (seminomas, and non-seminomas such as teratomas and choriocarcinomas), stromal tumors, germ cell tumors, thyroid cancer (including thyroid adenocarcinoma and medullary carcinoma) and urothelial cancer.

In some embodiments, cancer is selected from the group consisting of colorectal cancer, stomach cancer, liver cancer, prostate cancer, breast cancer, endometrial cancer, and ovarian cancer.

In another embodiment, cancer is a non-solid cancer. Examples of non-solid tumors include but are not limited to hematological neoplasms. As used herein, a hematologic neoplasm is a term of art which includes lymphoid disorders, myeloid disorders, and AIDS associated leukemias.

Lymphoid disorders include but are not limited to acute lymphocytic leukemia and chronic lymphoproliferative disorders (e.g., lymphomas, myelomas, and chronic lymphoid leukemias). Lymphomas include, for example, Hodgkin's disease, non-Hodgkin's lymphoma lymphomas, and lymphocytic lymphomas). Chronic lymphoid leukemias include, for example, T cell chronic lymphoid leukemias and B cell chronic lymphoid leukemias.

In a specific embodiment, cancer is selected from breast, carcinoid, cervical, colorectal, endometrial, glioma, head and neck, liver, lung, melanoma, ovarian, pancreatic, prostate, renal, gastric, thyroid and urothelial cancers.

In a specific embodiment, cancer is breast cancer. In a specific embodiment, cancer is carcinoid cancer. In a specific embodiment, cancer is cervical cancer. In a specific embodiment, cancer is colorectal cancer. In a specific embodiment, cancer is endometrial cancer. In a specific embodiment, cancer is glioma. In a specific embodiment, cancer is head and neck cancer. In a specific embodiment, cancer is liver cancer. In a specific embodiment, cancer is lung cancer. In a specific embodiment, the cancer is melanoma. In a specific embodiment, the cancer is ovarian cancer. In a specific embodiment, the cancer is pancreatic cancer, In a specific embodiment, cancer is prostate cancer. In a specific embodiment, cancer is renal cancer, In a specific embodiment, the cancer is gastric cancer. In a specific embodiment, cancer is thyroid cancer. In a specific embodiment, cancer is urothelial cancer.

In another specific embodiment, cancer is selected from the group consisting of: leukemia and multiple myeloma.

In one embodiment, a subject has previously received at least one prior therapeutic treatment, and has progressed subsequent to the administration of the at least one prior therapeutic treatment and prior to administration of a therapeutic agent. In one embodiment, a prior therapeutic treatment is selected from the group consisting of chemotherapy, immunotherapy, radiation therapy, stem cell transplant, hormone therapy, and surgery.

### Formulations

The present disclosure also provides pharmaceutical compositions comprising a compound disclosed herein, or a pharmaceutically acceptable salt and solvate thereof, and at least one pharmaceutically acceptable carrier, diluent, excipient and/or adjuvant.

The present disclosure also provides a medicament comprising at least one compound disclosed herein, or a pharmaceutically acceptable salt and solvate thereof, as active ingredient.

Generally, for pharmaceutical use, a compound disclosed herein may be formulated as a pharmaceutical preparation comprising at least one compound disclosed and at least one pharmaceutically acceptable carrier, diluent, excipient and/or adjuvant, and optionally one or more further pharmaceutically active compounds. Details regarding the presence of further pharmaceutically active compounds are provided hereafter.

By means of non-limiting examples, such a formulation may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. Formulations can optionally contain other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc. Compositions may also be formulated so as to provide rapid, sustained or delayed release of the active compound(s) contained therein.

Pharmaceutical preparations of the disclosure are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use.

Depending on the condition to be prevented or treated and the route of administration, a compound disclosed may be administered as a single daily dose, divided over one or more daily doses, or essentially continuously, e.g. using a drip infusion.

Another object of this disclosure is the use of the combination as a medicament, i.e. for medical use. Thus, in one embodiment, the disclosure provides the use of the combination of the disclosure for the manufacturing of a medicament. Especially, the disclosure provides the use of the combined pharmaceutical composition of the disclosure or the kit of the disclosure for the manufacturing of a medicament.

### EXEMPLIFICATION

### Example 1—Dose Escalation of Inupadenant

A Phase I portion of an ongoing multi-center, first-in-human, clinical trial to evaluate safety/tolerability, pharmacokinetic, pharmacodynamic and anti-tumor activity of inupadenant in adult patients with solid tumors who have exhausted standard treatment options was conducted. In addition, tumor biomarkers, including adenosine-pathway markers by immunohistochemistry (IHC), are being evaluated.

Results: Overall, 42 patients (21 patients in the dose escalation and an additional 21patients in a monotherapy expansion) with a median of 3 prior regimens were treated as of the data cut off. The dose levels investigated, along with the most frequent (>15%) treatment-emergent adverse events (TEAEs) across all dose levels are presented in Table 1. There were 7 AEs leading to discontinuation, of which 2 (atrial fibrillation and myocardial infarction) were considered to be possibly related to study drug by the investigator. No dose reductions were required. Two partial responses (PRs) were reported in patients with melanoma and prostate cancer. The patient with melanoma (NRAS-mutant) had received prior immunotherapy treatment with pembrolizumab and ipilumimab, and the patient with prostate cancer had received antiandrogen therapy and 2 prior lines of chemotherapy. At the date cut-off, both PRs were ongoing with a duration of response >230days. Stable disease as best response was observed in 12 patients and prolonged SD (>6 months) was observed in 3 patients with head & neck cancers, and non-small cell lung cancer. Response and stable disease were associated with a higher number of cells expressing the A2A receptor within the tumor at baseline, as measured by IHC.

Conclusions: Inupadenant monotherapy was generally well-tolerated as of the date cut-off at a dose of 80 mg twice daily with initial evidence of clinical benefit, including 2 durable partial responses in patients who have exhausted standard treatment options. Analysis of pre-treatment tumor biopsies has identified the A2A receptor as a biomarker which may be associated with clinical benefit.

TABLE 1
Most frequent TEAEs (>15%) in dose escalation and monotherapy expansion

| | Patients N(%) | | | | | |
|---|---|---|---|---|---|---|
| | 20 mg | 40 mg | 40 mg | 80 mg | 160 mg | |
| | QD | QD | BID | BID | BID | Total |
| Preferred Term | (N = 3) | (N = 3) | (N = 3) | (N = 27) | (N = 6) | (N = 42) |
| Fatigue | 1 (33.3) | 2 (66.7) | 1 (33.3) | 9 (33.3) | 4 (66.7) | 17 (40.5) |
| Anemia | 0 (0) | 2 (66.7) | 0 (0) | 11 (40.7) | 1 (16.7) | 14 (33.3) |
| Decreased appetite | 2 (66.7) | 1 (33.3) | 1 (33.3) | 6 (22.2) | 3 (50.0) | 13 (30.9) |
| Constipation | 2 (66.7) | 1 (33.3) | 0 (0) | 7 (25.9) | 1 (16.7) | 11 (26.2) |
| Aspartate amino- | 1 (33.3) | 1 (33.3) | 0 (0) | 4 (14.8) | 2 (33.3) | 8 (19.0) |
| transferase increased | | | | | | |
| Alanine amino- | 2 (66.7) | 0 (0) | 0 (0) | 3 (11.1) | 2 (33.3) | 7 (16.7) |
| transferase increased | | | | | | |
| Diarrhea | 0 (0) | 1 (33.3) | 1 (33.3) | 4 (14.8) | 1 (16.7) | 7 (16.7) |
| Nausea | 1 (33.3) | 1 (33.3) | 1 (33.3) | 3 (11.1) | 1 (16.7) | 7 (16.7) |
| Vomiting | 1 (33.3) | 0 (0) | 1 (33.3) | 4 (14.8) | 1 (16.7) | 7 (16.7) |
| Dyspnoea | 0 (0) | 0 (0) | 1 (33.3) | 3 (11.1) | 3 (50.0) | 7 (16.7) |

### Example 2—Assessment of A_{2A}R Expression in Clinical Tumor Samples Collected

Samples from the patients of Example 1 were collected. Baseline formalin-fixed paraffin-embedded biopsies, collected 1-28 days before inupadenant initiation (SCR), were sectioned at 4μm, and assessed for A_{2A}R expression at RNA and protein level using Nanostring technology and immunohistochemistry, respectively. For each subject, 1-3 biopsies were collected, analyzed separately, and then averaged. Tumor assessment was performed every 8 weeks, and tumor responses were evaluated using RECIST or PCWG3 criteria. Progression free survival (PFS) was calculated from first inupadenant dose to disease progression (event) or last patient visit (censored).

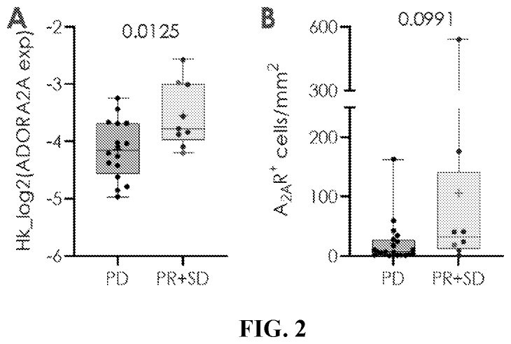

Baseline ADORA2A expression (FIG. 2A) and density of A_{2A}R⁺ cells within the tumor area (FIG. 2B) in subjects who had partial response or non-progressive disease (PR+SD, red and blue dots, respectively) as best response to inupadenant monotherapy, compared to patients who experienced progressive disease (PD, grey dots). Each dot represents the mean of available biopsies. Box plot showing median and quartiles, with whiskers from min to max. ' is the mean. P from t test (A) or Mann-Whitney (B), as appropriate.

For gene expression analysis, RNA was extracted from macrodissected tumor areas using High Pure FFPET RNA extraction kit and quantified using Quant-iT RiboGreen RNA Reagent and Kit. Total RNA (100 ng) was assayed using a customized nCounter PanCancer IO360 panel, and proprietary Nanostring gene signature scores were calculated. ADORA2A was analyzed using QCed, normalized data. Alternatively, evaluation of ADORA2A gene expression levels may be assessed using nCounter gene expression panels, unique probe pairs for ADORA2A as well as read counts based on RNA sequencing methods.

For protein expression analysis, sections were stained by IHC with a mouse monoclonal a-A_{2A}R antibody (clone 7F6-G5-A2, Novus Biologicals, diluted 1:750)) on a Ventana Discovery Ultra. Stained slides were scanned and analyzed with Visiopharm software to determine the density of A_{2A}R⁺ cells (cells/mm²) in the tumor areas. The receiver operating characteristic (ROC) curve analysis was used to determine optimal cut-off for baseline density of A_{2A}R⁺ cells and best response to inupadenant. The selected cut-off is 18 A_{2A}R⁺ cells/mm². Patients were grouped based on the optimal cut-off for baseline A_{2A}R⁺ cell density, PFS was estimated using the Kaplan-Meier method, and survival distributions were compared using the log-rank test. The best percent change from baseline size of target lesions was also calculated. Evaluation of A_{2A} receptor expression may be assessed using chromogenic or fluorescent immunohistochemistry staining of paraffin-embedded or frozen tissues sections of specimens embedded with paraffin wax, resins or cryo-embedding media (OCT or similar).

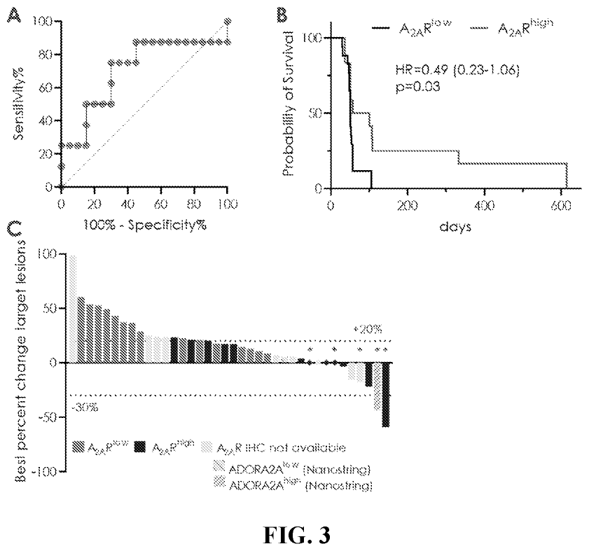

As shown in FIG. 3A, ROC curve for baseline density of A_{2A}R⁺ cells within the tumor area and best response to inupadenant (n=28). The area under the curve (AUC) is 0.71 (95% confidence interval: 0.47-0.94). With the selected cut-off, sensitivity is 75% and specificity is 70%.

As shown in FIG. 3B, Survival analysis by A_{2A}R⁺ cell density using the optimized cut-off. Hazard ratio (HR) with 95% confidence interval and p value from Log-rank test (n=29) for progression free survival are shown.

As shown in FIG. 3C, Waterfall plot of best percent change in the sum of target lesion diameters in RECIST-evaluable subjects (n=38). Green: subjects with low baseline tumor A_{2A}R; dark blue: subjects with high A_{2A}R; light blue: A_{2A}R data unavailable. For two patients with unavailable A_{2A}R data, gene expression levels below (low) or above (high) the mean ADORA2A levels assessed by Nanostring are indicated. * progression-free>6 months.

### Example 3—Assessment of A_{2A}R Expression in Human Cell Lines

A_{2A}Rexpression was analyzed on human cell lines, including cell lines of tumor origin (purchased from ATCC and DSMZ) and HEK-293 overexpressing A_{2A}R or A_{2B} (hA_{2A}R HEK-293 and hAzBR HEK-293, respectively, both from Perkin Elmer), using quantitative reverse transcription PCR (RT-qPCR), western blot and flow cytometry.

The same methods can be applied to RNA and proteins extracted to any patient-derived tissue material, including tumor resections, biopsies or aspirates.

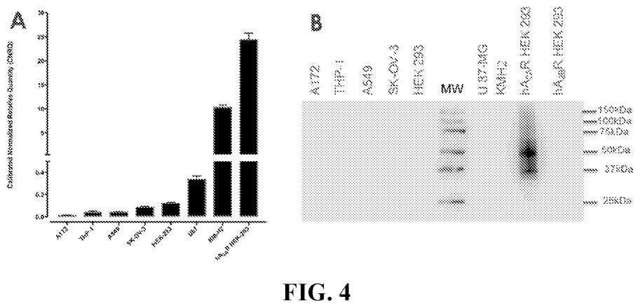

As shown in FIG. 4A, ADORA2A expression was analyzed by qRT-PCR in six human tumor cell lines, and HEK-293 wild-type or overexpressing A_{2A}R. Each bar represents the mean CNRQ (Calibrated Normalized Relative Quantity). The experiment was performed in triplicate.

As shown in FIG. 4B, A_{2A}R expression was assessed by Western blot in six human tumor cell lines, and HEK-293 wild-type or overexpressing A_{2A}R, or A_{2B}R as a control.

A_{2A}R expression was analyzed on HEK-293 wild-type or overexpressing A_{2A}R (hA_{2A}R HEK-293), using flow cytometry. Flow cytometry allowed detection of A_{2A}R using antibodies recognizing intracellular as well as extracellular A_{2A}R domains, and using probes targeting A_{2A}R .

The same methods can be applied to human cells isolated from fresh or frozen tumor samples and to blood cells.

Cells grown in the appropriate medium up to subconfluent state, were harvested and washed once with phosphate buffer saline (PBS). Total RNA was extracted from 2 millions cell pellets (RNeasy Plus Mini Kit, Qiagen) and eventual contaminating DNA was removed using Turbo DNA-free kit (Ambion). After quantification by Nanodrop one (Thermo Scientific, Isogen Lifescience), 2 μg of RNA were reverse transcribed into cDNA (RevertAid RT kit, Thermo Scientific). The qPCR was performed using human ADORA2A FAM-MGB primer/probe set (Hs00169123_ml/ 1571730, ThermoFisher Scientific) for the ADORA2A gene and human POLR2A FAM-MGB and human SDHA FAM-MGB primer/probe sets (Hs00172187_ml/160358 and Hs00417200_ml/ 1506652, both from ThermoFisher Scientific) on a LightCycler 96machine (Roche) with 20 ng of cDNA per well. Data were analyzed using the software “qbase+” (Biogazelle), and the Calibrated Normalized Relative Quantity (CNRQ) values were thereby calculated. Normalization was done with 2 housekeeping genes (POLR2A and SDHA).

Two millions of cells were collected and incubated for 30 min with RIPA lysis buffer (R0278, Sigma-Aldrich) and protease inhibitor (8159680747, Thermo Scientific) on ice. After centrifugation at 13,000 rpm (10 min, 4° C.), supernatants were collected and protein concentration was determined with a BCA Protein Assay Kit (23227, Thermo Scientific). Five (hA_{2A}R HEK-293) or twenty micrograms (other cell lines) of proteins were boiled at 95° C. (5 mins) with Laemmli buffer (complemented with 10% fresh beta-mercaptoethanol). After separation through electrophoresis, proteins were transferred on a nitrocellulose membrane. Membrane was then blocked for one hour with TBST 5% non-fat milk at RT. The primary mouse monoclonal a-A_{2A}R antibody (clone 7F6-G5-A2, Novus Biologicals, diluted 1:1000 in TBST 1% non-fat milk) was incubated overnight at 4° C. After three washes of the membrane, the secondary a-mouse HRP antibody (7076S, Cell Signaling Technology, diluted 1:1000) was incubated for 1 hour at RT. After three washes, the membrane was revealed by adding ECL substrate (170-5060, Bio-Rad) and reading it in the Westburg LI_OCR machine.

For intracellular staining with the A_{2A}R antibody, HEK-293 wt and hA_{2A}R HEK-293 cells were washed, fixed and permeabilized following supplier's recommendations (Foxp3/Transcription Factor Staining Buffer Set, eBioscience) and stained for 30 minutes at 4 C using the AF647-conjugated mouse monoclonal a-A_{2A}R antibody (clone 7F6-G5-A2, Novus Biologicals, diluted 1:50). After washes, cells were resuspended in FACS buffer, acquired in a BD LSR Fortessa and analyzed using FlowJo v10.7.2.

For staining with the A_{2A}R probe (A647-conjugated EOS850), HEK-293 wt and hA_{2A}R HEK-293 were washed, and the A_{2A}R probe was added to the cells for 30 minutes at 4 C after (grey), before (violet), or without (pink) fixation and permeabilization with human FoxP3 buffer set (#560098, BD Bioscience). After washes, cells were resuspended in FACS buffer, acquired in a BD LSR Fortessa and analyzed using FlowJo v10.7.2.

### Example 4 - Assessment of A_{2A}R Expression in Non-Clinical Tumor Tissue Samples

A_{2A}R and ADORA2A expression was assessed on human tumor tissue samples using fluorescent IHC and in situ hybridization (ISH), as shown for chromogenic ISH (CISH).

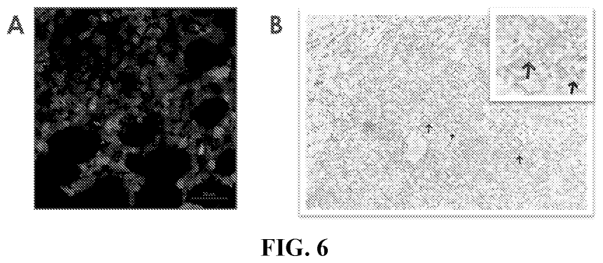

A_{2A}R ⁺ cells (FIG. 6A) and ADORA2A expression (FIG. 6B) in tumor tissue samples. A: Representative image of a tissue section co-stained with anti-A_{2A}R (green) and anti-CD3 (red) antibodies. B: Representative image of CISH on a tissue section. Arrows indicate ADORA2A RNA molecules.

### Immunofluorescence

After deparaffinization, sections of human tumor tissue (4-5 um) were incubated with the slides were incubated with CCI buffer (#06414575001, Roche, pH 9) at 95° C. for 40 minutes for antigen retrieval. After inhibition of endogenous peroxidases for 8 minutes and blocking of aspecific sites for 4 minutes, primary antibodies (mouse monoclonal a-A_{2A}R antibody, clone 7F6-G5-A2, Novus Biologicals, diluted 1:500 and prediluted anti-CD3 (2GV6) rabbit monoclonal Antibody, #5278422001, Ventana) were incubated for 1 hour at 37° C., followed by 1hour incubation at RT with secondary antibodies (Alexa Fluor 488-conjugated goat anti-rabbit IgG (H+L) Highly Cross-Adsorbed Secondary Antibody, #A11034 from Thermo Fisher, diluted 1:200, and Alexa Fluor 568-conjugated goat anti-mouse IgG (H+L) Highly Cross-Adsorbed Secondary Antibody, #A11031 from Thermo Fisher, diluted 1:1000). After slide cleaning, DAPI was added for 5 minutes. Results of the immunofluorescence are shown in FIG. 6A.

### Chromogenic In Situ Hybridization (CISH)

A_{2A} receptor RNA expression was evaluated by CISH using RNAscope 2.5 LS Red Assay on Leica Bond RX platform. Tumor tissues were validated for mRNA quality and accessibility using dapB negative probe and Hs-PPIB positive probe before applying A2A probe. ADORA2A stained slides and controls were digitalized with Nanozoomer (HAMAMATSU). Specimens stained with ADORA_{2A} ISH were scored by a pathologist using RNAscope scoring system with a semi-quantitative score ranging from 0 to 4 based on the amount of RNA signal (dots) visualized in the cells. Three-four fields were analy'sed for each section. For each section, H-score was also calculated using RNAscope scoring guidelines and HALO software. Results of the CISH are shown in FIG. 6B.

We claim:

1. A method of treating cancer characterized by increased A_{2A}R expression or density of A_{2A}R⁺ cells in a subject in need thereof, comprising administering to the subject a therapeutically effective amount of an adenosine receptor antagonist.

2. A method of treating cancer in a subject in need thereof, comprising administering to the subject a therapeutically effective amount of an adenosine receptor antagonist, wherein the subject has previously been identified as having increased A_{2A}R expression or density of A_{2A}R⁺ cells.

3. A method of treating a disease or disorder in a subject in need thereof, comprising administering to the subject a therapeutically effective amount of an adenosine receptor antagonist, wherein the subject has previously been identified as having increased A_{2A}R expression or density of A_{2A}R⁺ cells.

4. A method of treating cancer in a patient in need thereof, comprising selecting a patient with cancer having a diagnosis of increased level of A_{2A}R expression or density of A_{2A}R⁺ cells; and
treating the patient with an adenosine receptor antagonist.

5. A method of selecting a subject with cancer for treatment with an adenosine receptor antagonist, comprising
detecting the level of A_{2A}R expression or the density of A_{2A}R⁺ cells in a sample from the subject;
comparing the level of A_{2A}R expression or the density of A_{2A}R⁺ cells with a reference level or density;
selecting the subject for treatment with an adenosine receptor antagonist based on the comparison at the previous step.

6. The method of claim 5, wherein the subject is selected for treatment when A_{2A}R expression or density of A_{2A}R⁺ cells is increased.

7. The method of claim 5 or 6, wherein the sample is a tumor tissue sample.

8. Adenosine receptor antagonist, for use in the treatment of cancer in a patient in need thereof, wherein the cancer is characterized by increased A_{2A}R expression or density of A_{2A}R⁺ cells.

9. The method of any one of claim 1-4 or 6-7, or the adenosine receptor antagonist for use according to claim 8, wherein A_{2A}R expression or density of A_{2A}R⁺ cells is increased by comparison to a reference level or density determined in a sample from a subject not affected and/or diagnosed with cancer.

10. The method of any one of claim 1-4 or 6-7, or the adenosine receptor antagonist for use according to claim 8, wherein the level of A_{2A}R expression or density of A_{2A}R⁺ cells is increased by comparison to a reference level or density determined in a non-cancerous sample from the same subject.

11. The method or adenosine receptor antagonist for use according to any one of claims 1-10, wherein the level of A_{2A}R expression is measured using gene expression profiling.

12. The method or adenosine receptor antagonist for use according to any of claims 1-11, wherein the level of A_{2A}R expression or density of A_{2A}R⁺ cells is measured using an assay selected from the group consisting of Nanostring technology, immunohistochemistry (IHC), quantitative reverse transcription PCR (RT-qPCR), Western blot, flow cytometry, fluorescent IHC, and in situ hybridization.

13. The method or adenosine receptor antagonist for use according to claim 12, wherein the assay is Nanostring technology.

14. The method or adenosine receptor antagonist for use according to claim 12, wherein the assay is immunohistochemistry (IHC).

15. The method or adenosine receptor antagonist for use according to claim 12, wherein the assay is RT-qPCT.

16. The method or adenosine receptor antagonist for use according to claim 12, wherein the assay is Western blot.

17. The method or adenosine receptor antagonist for use according to claim 12, wherein the assay is flow cytometry.

18. The method or adenosine receptor antagonist for use according to claim 12, wherein the assay is fluorescent IHC.

19. The method or adenosine receptor antagonist for use according to claim 12, wherein the assay is in situ hybridization.

20. The method or adenosine receptor antagonist for use according to any of claims 1-19, wherein the A_{2A}R log2 expression value relative to the reference value is greater than about 3.95.

21. The method or adenosine receptor antagonist for use according to any of claims 1-19, wherein an increased density of A_{2A}R⁺ cells is equal to or above about 13 A_{2A}R⁺ cells/mm².

22. The method or adenosine receptor antagonist for use according to any of claims 1-19, wherein an increased density of A_{2A}R⁺ cells is equal to or above about 18 A_{2A}R⁺ cells/mm².

23. The method or adenosine receptor antagonist for use according to any of claims 1-22, wherein the adenosine receptor antagonist is an A_{2A}R antagonist.

24. The method or adenosine receptor antagonist for use according to any of claims 1-23, wherein the adenosine receptor antagonist is selected from the group consisting of
(R, S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one,
or a pharmaceutically acceptable salt thereof.

25. The method or adenosine receptor antagonist for use according to any of claims 1-24, further comprising administration of an additional therapeutic agent.

26. The method or adenosine receptor antagonist for use according to claim 25, wherein the adenosine receptor antagonist is administered prior to the additional therapeutic agent.

27. The method or adenosine receptor antagonist for use according to claim 25, wherein the adenosine receptor antagonist is administered simultaneously with the additional therapeutic agent.

28. The method or adenosine receptor antagonist for use according to claim 25, wherein the adenosine receptor antagonist is administered after the additional therapeutic agent.

29. The method or adenosine receptor antagonist for use according to any of claims 1-28, wherein the subject has previously received treatment with an additional therapeutic agent.

30. The method or adenosine receptor antagonist for use according to any one of claims 1-29, wherein the cancer is selected from the group consisting of lung cancer, endometrial cancer, gastric cancer, melanoma breast cancer, colorectal cancer, oral squamous cell carcinoma, and a head/neck cancer.

31. The method or adenosine receptor antagonist for use according to any one of claims 1-29, wherein the cancer is selected from the group consisting of melanoma, prostate cancer, head & neck cancers, and non-small cell lung cancer.

32. The method of any of claims 1-30 wherein the A_{2A}R⁺ cells are TIL.


SEQUENCE LISTING (7 sequences)

SEQ ID NO: 1 - protein, 412 aa, Homo sapiens
MPIMGSSVYITVELAIAVLAILGNVLVCWAVWLNSNLQNVTNYFVVSLAAADIAVGVLAIPFAITISTGFCAACHGCLFIACFVLVLTQSSIFSLLAIAIDRYIAIRIPLRYNGLVTGTRAKGIIAICWVLSFAIGLTPMLGWNNCGQPKEGKNHSQGCGEGQVACLFEDVVPMNYMVYFNFFACVLVPLLLMLGVYLRIFLAARRQLKQMESQPLPGERARSTLQKEVHAAKSLAIIVGLFALCWLPLHIINCFTFFCPDCSHAPLWLMYLAIVLSHTNSVVNPFIYAYRIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVS

SEQ ID NO: 2 - protein, 9 aa, Artificial Sequence
  source: /note=“Description of Artificial Sequence Synthetic peptide”
YTFTSYYMH

SEQ ID NO: 3 - protein, 17 aa, Artificial Sequence
  source: /note=“Description of Artificial Sequence Synthetic peptide”
VIGPSGASTSYAQKFQG

SEQ ID NO: 4 - protein, 14 aa, Artificial Sequence
  source: /note=“Description of Artificial Sequence Synthetic peptide”
ARDHSDYWSGIMEV

SEQ ID NO: 5 - protein, 12 aa, Artificial Sequence
  source: /note=“Description of Artificial Sequence Synthetic peptide”
RASQSVRSSYLA

SEQ ID NO: 6 - protein, 7 aa, Artificial Sequence
  source: /note=“Description of Artificial Sequence Synthetic peptide”
GASSRAT

SEQ ID NO: 7 - protein, 9 aa, Artificial Sequence
  source: /note=“Description of Artificial Sequence Synthetic peptide”
QQYFSPPWT